(19) United States
(12) Patent Application Publication
(10) Pub. No.: US 2018/0273469 A1
(43) Pub. Date: Sep. 27, 2018

(54) MARMELIN ANALOGS AND METHODS OF USE IN CANCER TREATMENT

(71) Applicant: The University of Kansas, Lawrence, KS (US)

(72) Inventors: Shrikant Anant, Shawnee, KS (US); Dharmalingam Subramaniam, Olathe, KS (US); Subhash Padhye, Erawana (IN)

(21) Appl. No.: 15/996,210

(22) Filed: Jun. 1, 2018

Related U.S. Application Data
(62) Division of application No. 15/315,521, filed on Dec. 1, 2016.
(60) Provisional application No. 62/008,350, filed on Jun. 5, 2014.

(51) Int. Cl. C07C 243/38; C07C 243/18; C07D 209/18; C07D 307/68; C07D 213/82; C07C 49/245; C07D 311/22; C07D 311/12; C07D 213/81

(52) U.S. Cl. CPC C07C 243/38; C07C 243/18; C07D 209/18; C07D 307/68; C07D 213/82; C07D 213/81; C07C 49/245; C07D 311/22; C07D 311/12

(57) ABSTRACT

A pharmaceutical composition can include: a marmelin analog compound, and a pharmaceutically acceptable carrier having the compound. The compound can be present in a therapeutically effective amount to treat or inhibit a disease state. The disease state can be cancer. The cancer can be selected from brain cancers, head and neck cancers, thyroid cancers, gastrointestinal cancers, esophageal cancers, stomach cancers, pancreatic cancers, liver cancers, colo-rectal cancers, lung cancers, kidney cancers, prostate cancers, bladder cancers, testicular cancers, breast cancers, ovarian cancers, cervical cancers, and melanomas. The carrier includes a cyclodextrin, which may form a complex with the compound. The compounds and compositions can be used to treat or inhibit progression of cancers. Colo-rectal, bladder, and prostate cancers are examples of some of the cancers that can be treated with the marmelin analog compounds.

### CROSS-REFERENCE

This patent application is a divisional application of U.S. application Ser. No. 15/315,521 filed Dec. 1, 2016, which is a section 371 Nationalization of International Application No. PCT/US2015/034530 filed Jun. 5, 2015, which claims the benefit of U.S. Provisional Patent Application 62/008,350 filed Jun. 5, 2014, which applications are each incorporated herein by specific reference in their entirety.

### GOVERNMENT SUPPORT

This invention was made with government support under CA109269 and CA135559 and CA182872 awarded by the National Institute of Health. The government has certain rights in the invention.

### BACKGROUND

Colorectal cancer is a second leading cause of adult cancer related death in the Unites States, and is associated with a high mortality rate. The lifetime risk of developing colorectal cancer in both men and women is about 1 in 20 (5.1%). The American Cancer Society (ACS) estimated 102,480 new cases (50, 920 men and 52, 390 women) would be diagnosed with colon cancer during 2013 and also estimated 50,830 deaths (26,300 men and 24,530). Current therapy for colorectal cancer is surgical resection, chemotherapy and radiation. Current chemotherapy includes 5-flurouracil, oxaliplatin, Irinotecan hydroloride or drug combinations FOLFOX or FOLFIRI. Because the conventional therapies, including surgical resection, chemotherapy, and radiation are often inadequate in treating this disease and result in severe side effects, new treatment options are critically needed. Despite the emergence of novel targeted agents and the use of various therapeutic combinations, no treatment options are available that are curative in patients with advanced cancer.

The magnitude of this problem mandates the need for novel therapeutic agents.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 includes images that show analog compounds inhibit colony formation by colon cancer cells.

FIG. 2 includes images of western blot gels that show an analog compound is a potent apoptosis inducer in colon cancer cells.

FIG. 3 includes images of western blot gels that show an analog compound inhibits cancer promoting genes.

FIGS. 4A and 4B includes graphs that shows analog compounds inhibit tumor volume increases over time compared to a control.

FIG. 4C includes images that show analog compounds inhibited tumor growth.

FIG. 5 includes images that show analog compounds inhibit colonosphere formation.

FIG. 6 includes images of western blot gels that show an analog compound inhibits expression of DCLK1, LGR5, and CD44.

FIG. 7 includes graphs that show analog compounds inhibit DCLK1 positive stem cells.

FIGS. 8A-8B include graphs that show analog compounds inhibit DCLK1 kinase activity.

FIG. 9 includes images of western blot gels that show an analog compound inhibits expression of Notch 1, Jagged 1, and Hes 1.

FIG. 10 includes images of western blot gels that show an analog compound inhibits γ-secretase complex proteins.

FIG. 11 includes images of western blot gels that show an analog compound inhibits phosphorylation of Mst1/2, LATS1/2, and YAP1.

FIG. 12 includes images of western blot gels that show an analog compound inhibits expression of TEAD 1, TEAD 2, and TEAD 4.

FIG. 13 includes images that show analog compounds inhibit colony formation by colon cancer cells.

FIG. 14 includes images that show analog compounds inhibit colony formation by pancreatic cancer cells.

FIG. 15 includes images that show analog compounds inhibit colonosphere formation.

FIGS. 16A-16B include graphs that show analog compounds inhibit tumor growth.

FIG. 16C includes an images that shows analog compounds inhibit tumor growth.

FIG. 17 includes images that show analog compounds inhibit colony formation by colon cancer cells.

FIG. 18 includes images that show analog compounds inhibit colonosphere formation.

FIGS. 19A-19B include graphs that show analog compounds inhibit tumor growth.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention relates to compounds that can used for treating or inhibiting the progression of cancer. The compounds are analogs of marmelin.

In one embodiment, the compounds of the invention can include the structure of Formula 1, Formula 2, Formula 3, Formula 4, and Formula 5 or any derivative thereof, prodrug thereof, salt thereof, or stereoisomer thereof, or having any chirality at any chiral center, or tautomer, polymorph, solvate, or combination thereof.

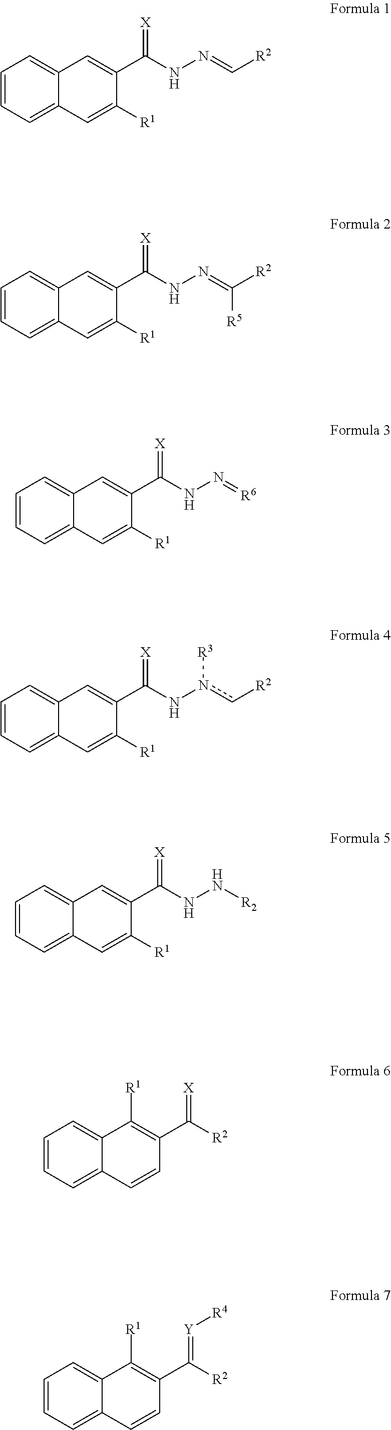

In any one of Formula 1, Formula 2, Formula 3, Formula 4, Formula 5, Formula 6, and Formula 7, R¹ or R² or R³ or R⁴ or R⁵ can be independently any substituent. As such, R¹ or R² or R³ or R⁴ or R⁵ can be a hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, derivatives thereof, substituted or unsubstituted, or combinations thereof as well as other well-known chemical substituents. R⁶ can be a substituted or unsubstituted cyclohexane, such as a cyclohexadienone, such as for example 2,6-di-tert-butylcyclohexa-2,5-dienone, and optionally, R² and R⁵ can cooperate to form such a substituted or unsubstituted cyclohexane (e.g., 2,6-di-tert-butylcyclohexa-2,5-dienone). R¹ or R² or R³ or R⁴ or R⁵ can be independently selected from the group of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano(—C≡N), isocyano (—N⁺≡C⁻), cyanato isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—S₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O—)), phospho (—PO₂), phosphino (—PH₂), derivatives thereof, and combinations thereof. The alkyl groups of these substituents can be short alkyls, such as C₁-C₁₂, C₁-C₁₁, C₁-C₁₀, C₁-C₉, C₁-C₈, C₁-C₇, C₁-C₆, C₁-C₅, C₁-C₄, C₁-C₃, or C₁-C₂, straight or branched and/or substituted or unsubstituted. For example R¹ or R² or R³ or R⁴ or R⁵ can each independently be methyl, ethyl, propyl, isopropyl, butyl, tertbutyl, pentyl, hexyl, cyclohexyl, benzyl, heptyl, and any configuration thereof, substituted or unsubstituted. X can be S, O, N, NH, or P. Y can be C, CH, or N. The dashed lines illustrate optional bonding where the nitrogen has only one of the dashed lines being a bond, such that when the dashed line from the nitrogen to R³ is a bond, then the other dashed lines is nothing, or alternatively when the dashed line from the nitrogen to the carbon linked to R² is a bond, the dashed line from the nitrogen to R³ is nothing and R³ is nothing.

In one embodiment, the compounds of the invention can include the structure of Formula 1, or any derivative thereof, prodrug thereof, salt thereof, or stereoisomer thereof, or having any chirality at any chiral center, or tautomer, polymorph, solvate, or combination thereof. In such an embodiment, X can be O, and R¹ can be a hydroxyl, halogen, or short alkyl. In one example R¹ can be —OH. In one aspect, R² can be as shown below:

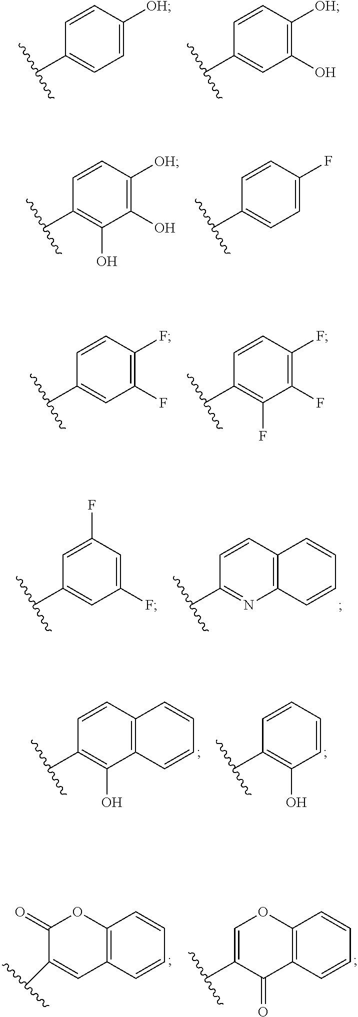

or the like.

In one embodiment, the compounds of the invention can include the structure of Formula 2, or any derivative thereof, prodrug thereof, salt thereof, or stereoisomer thereof, or having any chirality at any chiral center, or tautomer, polymorph, solvate, or combination thereof. In such an embodiment, X can be O, and R¹ can be a hydroxyl, halogen, or short alkyl. In one example R¹ can be —OH. In one aspect, R² can be as defined herein for Formula 1. In one aspect, R⁵ can be as defined for R². In one aspect, R⁵ can be any of the short alkyls, such as C₁-C₁₂, C₁-C₁₁, C₁-C₁₀, C₁-C₉, C₁-C₈, C₁-C₇, C₁-C₆, C₁-C₅, C₁-C₄, C₁-C₃, or C₁-C₂, straight or branched and/or substituted or unsubstituted. In one example, R⁵ is methyl. In another example R⁵ is hydrogen. In one aspect, R² and R⁵ can cooperate to form a substituted or unsubstituted cyclohexane (e.g., 2,6-di-tert-butylcyclohexa-2,5-dienone) ring structure.

In one embodiment, the compounds of the invention can include the structure of Formula 3, or any derivative thereof, prodrug thereof, salt thereof, or stereoisomer thereof, or having any chirality at any chiral center, or tautomer, polymorph, solvate, or combination thereof. In such an embodiment, X can be O, and R¹ can be a hydroxyl, halogen, or short alkyl. In one example R¹ can be —OH. In one aspect, R⁶ can be a substituted or unsubstituted cyclohexane, such as a cyclohexadienone, such as for example 2,6-di-tert-butylcyclohexa-2,5-dienone. For example, R6 can be:

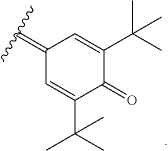 [SMILES: *=C1C=C(C(C)(C)C)C(=O)C(C(C)(C)C)=C1]

In one embodiment, the compounds of the invention can include the structure of Formula 4, or any derivative thereof, prodrug thereof, salt thereof, or stereoisomer thereof, or having any chirality at any chiral center, or tautomer, polymorph, solvate, or combination thereof. In such an embodiment, X can be O, and R¹ can be a hydroxyl, halogen, or short alkyl. In one example R¹ can be —OH. In one aspect, R² and R³ can be as defined herein for Formula 1. In one aspect, R³ can be as defined for R². In one aspect, R³ can be any of the short alkyls, such as C₁-C₁₂, C₁-C₁₁, C₁-C₁₀, C₁-C₉, C₁-C₈, C₁-C₇, C₁-C₆, C₁-C₅, C₁-C₄, C₁-C₃, or C₁-C₂, straight or branched and/or substituted or unsubstituted. In one example, R³ is methyl. In another example R³ is hydrogen. In one aspect, the dashed lines illustrate optional bonding where the nitrogen has only one of the dashed lines being a bond, such that when the dashed line from the nitrogen to R³ is a bond, then the other dashed lines is nothing, or alternatively when the dashed line from the nitrogen to the carbon linked to R² is a bond, the dashed line from the nitrogen to R³ is nothing and R³ is nothing.

In one embodiment, the compounds of the invention can include the structure of Formula 5, or any derivative thereof, prodrug thereof, salt thereof, or stereoisomer thereof, or having any chirality at any chiral center, or tautomer, polymorph, solvate, or combination thereof. In such an embodiment, X can be O, and R¹ can be a hydroxyl, halogen, or short alkyl. In one example 10 can be —OH. In one aspect, R² can be as defined herein for Formula 1. In one aspect, R² can be any of the short alkyls, such as C₁-C₁₂, C₁-C₁₁, C₁-C₁₀, C₁-C₉, C₁-C₈, C₁-C₇, C₁-C₆, C₁-C₅, C₁-C₄, C₁-C₃, or C₁-C₂, straight or branched and/or substituted or unsubstituted. In one example, R² is methyl. In another example R² is hydrogen.

In one embodiment, the compounds of the invention can include the structure of Formula 6, or any derivative thereof, prodrug thereof, salt thereof, or stereoisomer thereof, or having any chirality at any chiral center, or tautomer, polymorph, solvate, or combination thereof. In such an embodiment, X can be O, and R¹ can be a hydroxyl, halogen, or short alkyl. In one example R¹ can be —OH. In one aspect, R² can be as defined herein for Formula 1. In one aspect, R² can be any of the short alkyls, such as C₁-C₁₂, C₁-C₁₁, C₁-C₁₀, C₁-C₉, C₁-C₈, C₁-C₇, C₁-C₆, C₁-C₅, C₁-C₄, C₁-C₃, or C₁-C₂, straight or branched and/or substituted or unsubstituted. In one example, R² is methyl.

In one embodiment, the compounds of the invention can include the structure of Formula 7, or any derivative thereof, prodrug thereof, salt thereof, or stereoisomer thereof, or having any chirality at any chiral center, or tautomer, polymorph, solvate, or combination thereof. In one aspect, Y can be CH, or N. In an embodiment, Y can be CH, or N, and R¹ can be a hydroxyl, halogen, or short alkyl. In one example R¹ can be —OH. In one aspect, R² can be as defined herein for Formula 1. In one aspect, R² can be any of the short alkyls, such as C₁-C₁₂, C₁-C₁₁, C₁-C₁₀, C₁-C₉, C₁-C₈, C₁-C₇, C₁-C₆, C₁-C₅, C₁-C₄, C₁-C₃, or C₁-C₂, straight or branched and/or substituted or unsubstituted. In one example, R² is methyl. In one aspect, R³ can be as defined herein for Formula 1. In one aspect, R³ can be as shown below:

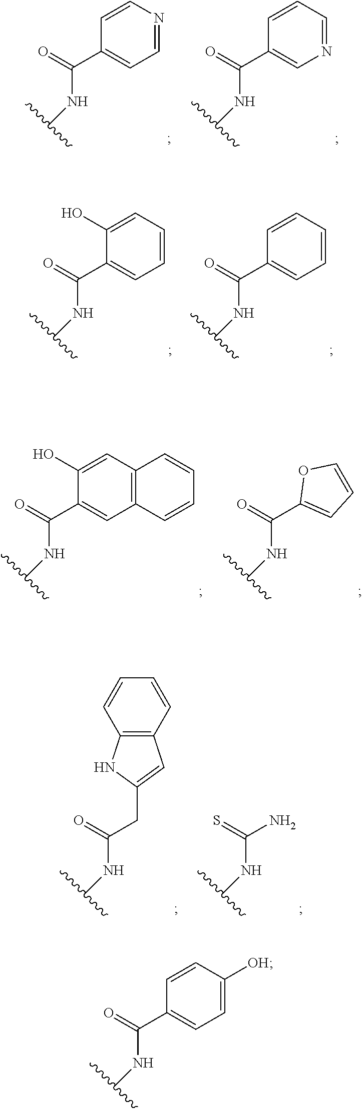

or the like.

In one embodiment, R² is one of:

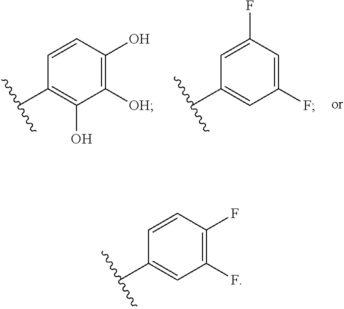 [SMILES: *c1ccc(O)c(O)c1O ; *c1cc(F)cc(F)c1 ; "or" ; *c1ccc(F)c(F)c1]

In one embodiment, wherein R² is:

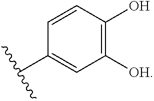 [SMILES: *c1ccc(O)c(O)c1]

In one embodiment, R² is not one of:

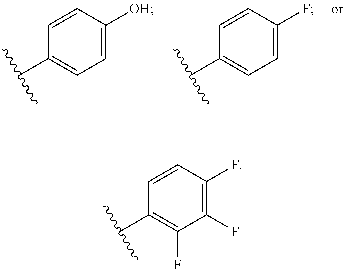 [SMILES: *c1ccc(O)cc1 ; *c1ccc(F)cc1 ; *c1ccc(F)c(F)c1F]

In any of the embodiments, X is O, and R¹ is hydroxyl.

In one embodiment, R² and/or R⁵ is a short alkyl. In one aspect, one of R² or R⁵ is hydrogen.

In one embodiment, R² and R⁵ cooperate to form a ring. In one aspect, the ring formed by R² and R⁵ is a substituted or unsubstituted cyclohexane. In one aspect, the ring formed by R² and R⁵ is a cyclohexadienone. In one aspect, the ring formed by R² and R⁵ is 2,6-di-tert-butylcyclohexa-2,5-dienone.

In one embodiment, R⁵ is hydrogen or a short alkyl. In one aspect, R² is one of:

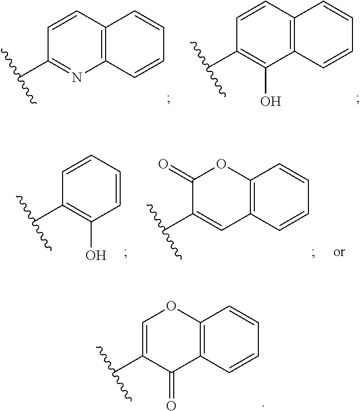

In one aspect, R² is one of:

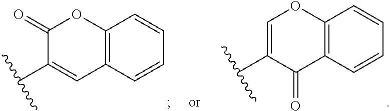 [SMILES: *c1cc2ccccc2oc1=O ; *c1coc2ccccc2c1=O]

In one aspect, R² is one of:

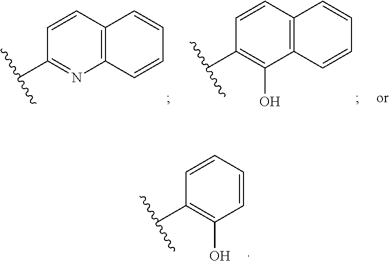

In one aspect, R² is not one of:

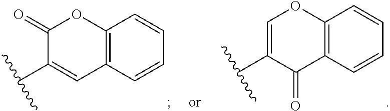 [SMILES: *c1cc2ccccc2oc1=O ; *c1coc2ccccc2c1=O]

In one aspect, the structure is Formula 1 or Formula 2 or Formula 3, or any derivative thereof, prodrug thereof, salt thereof, or stereoisomer thereof, or having any chirality at any chiral center, or tautomer, polymorph, solvate, or combination thereof.

In one embodiment, the structure is Formula 3, or any derivative thereof, prodrug thereof, salt thereof, or stereoisomer thereof, or having any chirality at any chiral center, or tautomer, polymorph, solvate, or combination thereof, and R⁶ is:

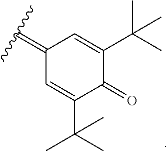 [SMILES: *=C1C=C(C(C)(C)C)C(=O)C(C(C)(C)C)=C1]

In one embodiment, the structure is Formula 4, or any derivative thereof, prodrug thereof, salt thereof, or stereoisomer thereof, or having any chirality at any chiral center, or tautomer, polymorph, solvate, or combination thereof, and R³ is hydrogen.

In one embodiment, the structure is Formula 5, or any derivative thereof, prodrug thereof, salt thereof, or stereoisomer thereof, or having any chirality at any chiral center, or tautomer, polymorph, solvate, or combination thereof, and R² is hydrogen.

In one embodiment, the structure is Formula 6, or any derivative thereof, prodrug thereof, salt thereof, or stereoisomer thereof, or having any chirality at any chiral center, or tautomer, polymorph, solvate, or combination thereof. In one aspect, R¹ is a hydroxyl. In one aspect, X is O. In one aspect, R² a short alkyl, such as methyl.

In one embodiment, the structure is not Formula 6.

In one embodiment, the structure is Formula 7, or any derivative thereof, prodrug thereof, salt thereof, or stereoisomer thereof, or having any chirality at any chiral center, or tautomer, polymorph, solvate, or combination thereof. In one aspect, R¹ is a hydroxyl. In one aspect, Y is N. In one aspect, R² is a short alkyl, such as methyl. In one aspect, R⁴ is one of:

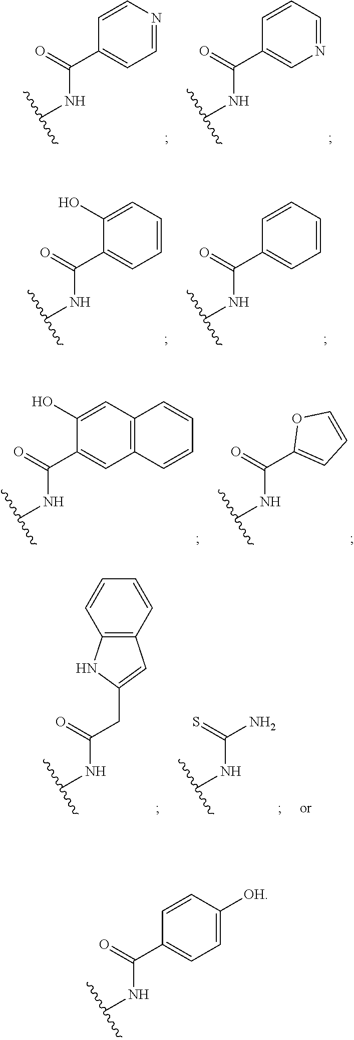

In one aspect, R⁴ is one of:

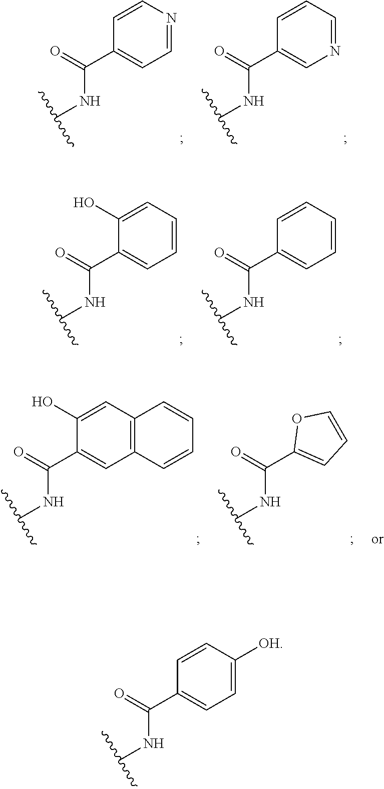

In one aspect, R⁴ is not one of:

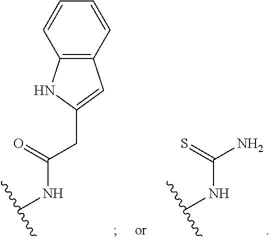 [SMILES: *NC(=O)Cc1cc2ccccc2[nH]1 ; *NC(N)=S]

In one embodiment, the compound has any one of the following structures or any derivative thereof, prodrug thereof, salt thereof, or stereoisomer thereof, or having any chirality at any chiral center, or tautomer, polymorph, solvate, or combination thereof:

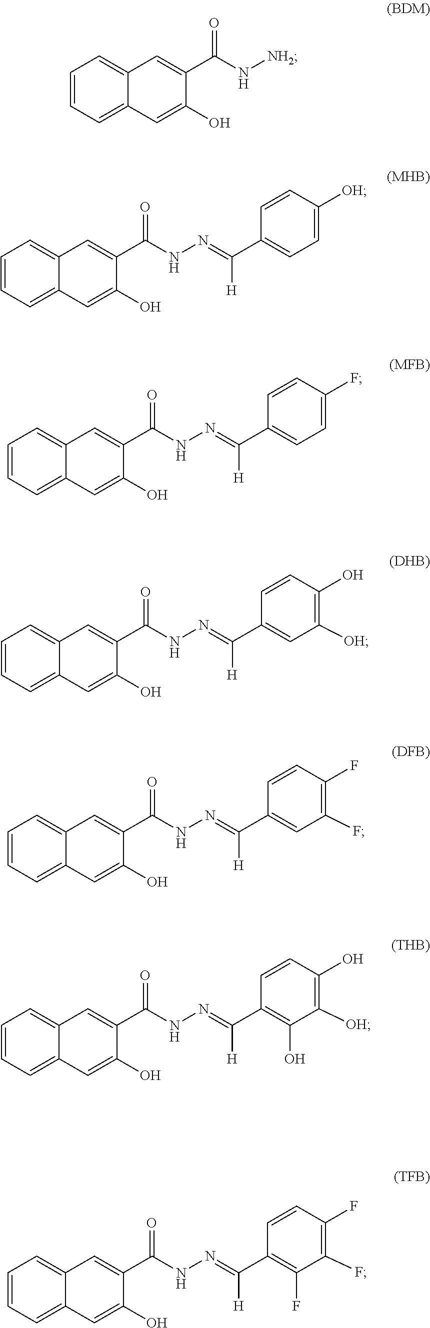

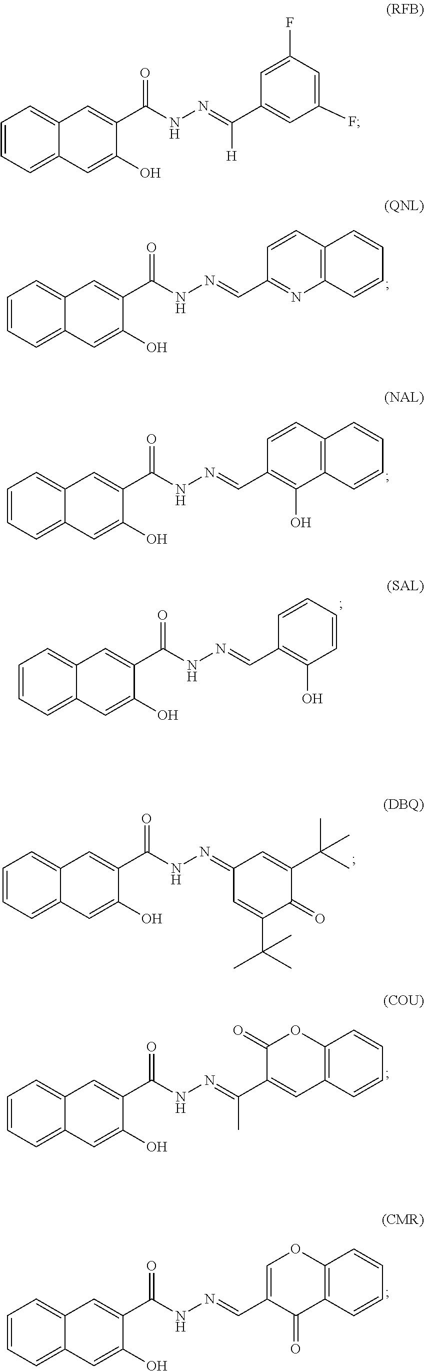

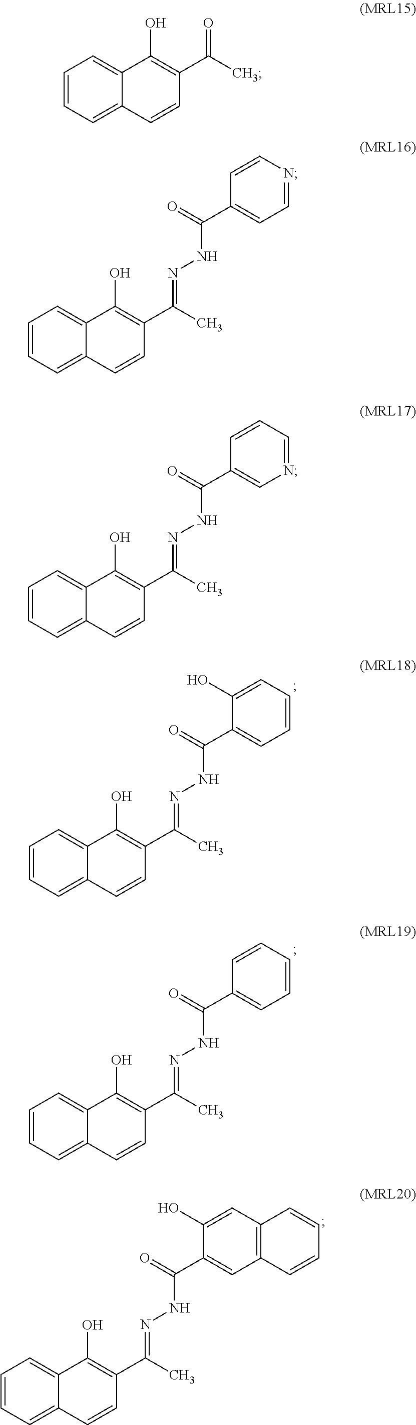 [SMILES: CC(=O)c1ccc2ccccc2c1O ; C/C(=N\NC(=O)c1ccncc1)c1ccc2ccccc2c1O ; C/C(=N\NC(=O)c1cccnc1)c1ccc2ccccc2c1O ; C/C(=N\NC(=O)c1ccccc1O)c1ccc2ccccc2c1O ; C/C(=N\NC(=O)c1ccccc1)c1ccc2ccccc2c1O ; C/C(=N\NC(=O)c1cc2ccccc2cc1O)c1ccc2ccccc2c1O]

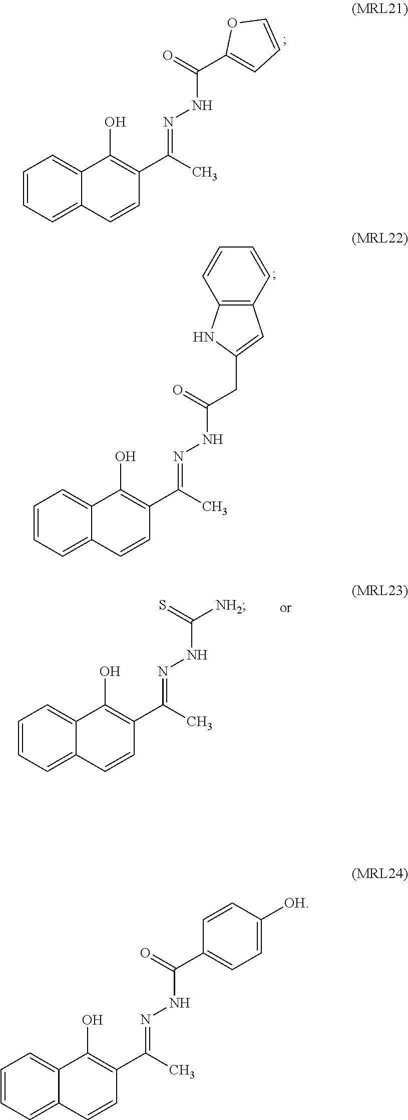 [SMILES: C/C(=N\NC(=O)c1ccco1)c1ccc2ccccc2c1O ; C/C(=N\NC(=O)Cc1cc2ccccc2[nH]1)c1ccc2ccccc2c1O ; C/C(=N\NC(N)=S)c1ccc2ccccc2c1O ; C/C(=N\NC(=O)c1ccc(O)cc1)c1ccc2ccccc2c1O]

The chemical structures for any of Formulae 1-7 can be prepared by routine chemistry based on the example structures provided herein.

In one embodiment, example chemical structures of Formula 1 can be prepared by Scheme 1 provided below. As a note, marmelin is considered to be Compound 1. Compound 2 and Compounds 3a-f are reagents that when reacted in concentrated hydrochloric acid and methanol at 60° C., Compounds 4a-4f are synthesized.

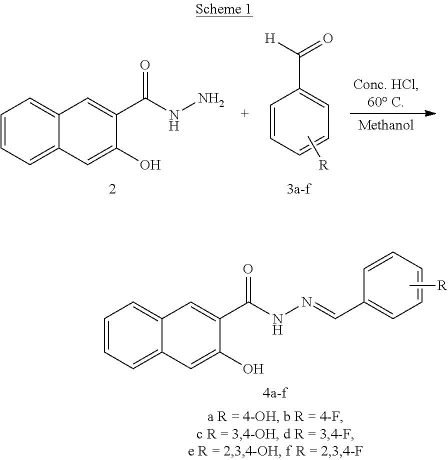

In one embodiment, example chemical structures of Formula 1 can be prepared by Scheme 2 provided below.

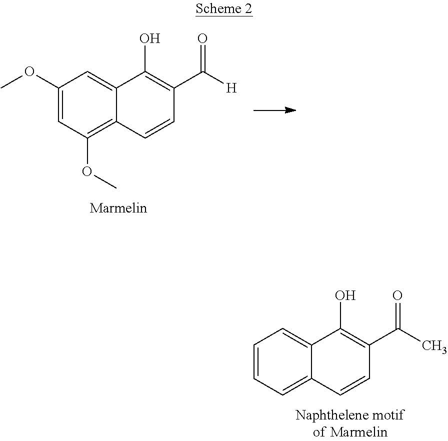 [SMILES: COc1cc(OC)c2ccc(C=O)c(O)c2c1 ; CC(=O)c1ccc2ccccc2c1O]

From Schemes 1 and 2 and the general knowledge of synthetic chemistry, any of the compounds described herein as represented by the figures can be synthesized.

The term “alkyl” or “aliphatic” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, or 1 to about 12 carbon atoms. The term “lower alkyl” intends an alkyl group of 1 to 6 carbon atoms. Substituents identified as “C₁-C₆ alkyl” or “lower alkyl” contains 1 to 3 carbon atoms, and such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The terms “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, or 2 to 12 carbon atoms. The term “lower alkenyl” intends an alkenyl group of 2 to 6 carbon atoms, and the specific term “cycloalkenyl” intends a cyclic alkenyl group, or having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, or 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein contain 1 to 3 carbon atoms, and such substituents contain 1 or 2 carbon atoms (i.e., methoxy and ethoxy).

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Examples of aryl groups contain 5 to 20 carbon atoms, and aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aryloxy” as used herein refers to an aryl group bound through a single, terminal ether linkage, wherein “aryl” is as defined above. An “aryloxy” group may be represented as —O-aryl where aryl is as defined above. Examples of aryloxy groups contain 5 to 20 carbon atoms, and aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Examples of aralkyl groups contain 6 to 24 carbon atoms, and aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethyinaphthyl, 7-cyclooctylnaphthyl, 3-ethyl-cyclopenta-1,4-diene, and the like.

The term “cyclic” refers to alicyclic or aromatic substituents that may or may not be substituted and/or heteroatom containing, and that may be monocyclic, bicyclic, or polycyclic.

The terms “halo” and “halogen” are used in the conventional sense to refer to a chloro, bromo, and fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

The term “hydrocarbyl” refers to univalent hydrocarbyl radicals containing 1 to about 30 carbon atoms, or 1 to about 24 carbon atoms, or 1 to about 18 carbon atoms, or about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, heteroatom-containing alkenyl, and heteroatom-containing aryl.”

In one embodiment, a pharmaceutical composition can include a compound of one of the embodiments, and a pharmaceutically acceptable carrier containing the compound. In one aspect, the compound is present in a therapeutically effective amount to treat or inhibit a disease state. In one aspect, the disease state is cancer. In one aspect, the cancer is selected from brain cancers, head and neck cancers, thyroid cancers, gastrointestinal cancers, esophageal cancers, stomach cancers, pancreatic cancers, liver cancers, colo-rectal cancers, lung cancers, kidney cancers, prostate cancers, bladder cancers, testicular cancers, breast cancers, ovarian cancers, cervical cancers, and melanomas. In one aspect, the cancer is selected from colon, pancreatic, and bladder cancers.

Pharmaceutical compositions can include the compounds of the invention, and can include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (e.g., Tween®), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1 (2,3-dioleyloxy)propyl)N,N,N-trimethylammonium chloride (DOTMA), diolesyl-phosphatidyl-ethanolamine (DOPE), and liposomes. Such compositions should contain a therapeutically effective amount of the compound, together with a suitable amount of carrier so as to provide the form for direct administration to the patient.

The compositions described herein can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration. Common carriers or excipients can be used for preparing pharmaceutical compositions designed for such routes of administration.

In one embodiment, a method for treating or inhibiting progression of cancer can include: providing a composition of one of the embodiments, and administering the composition to a subject having or susceptible to cancer. In one aspect, the subject having cancer or precancerous biological indicators, such as genes that increase the likelihood of developing cancer, such as BRACA genes. In one aspect, the cancer is selected from brain cancers, head and neck cancers, thyroid cancers, gastrointestinal cancers, esophageal cancers, stomach cancers, pancreatic cancers, liver cancers, colo-rectal cancers, lung cancers, kidney cancers, prostate cancers, bladder cancers, testicular cancers, breast cancers, ovarian cancers, cervical cancers, and melanomas. In one aspect, the cancer is selected from colon, pancreatic, and bladder cancers. In one aspect, the compound of the composition is administered in an effective amount to inhibit the action of transcription factor NF-κB. In one aspect, the compound of the composition is administered in an effective amount to bind into the active site of p50 subunit of NF-κB. In one aspect, the compound of the composition is administered in an effective amount to bind to the active site of DCLK1. In one aspect, the compound of the composition is administered in an effective amount to bind to inhibit cancer cell proliferation. In one aspect, the compound of the composition is administered in an effective amount to bind to cancer cell colony formation. In one aspect, the compound of the composition is administered in an effective amount to reduce tumor size. In one aspect, the compound of the composition is administered in an effective amount to inhibit tumor growth. In one aspect, the compound of the composition is administered in an effective amount to inhibit spheroid formation. In one aspect, the compound of the composition is administered in an effective amount to inhibit cancer stem cells from forming a tumor.

The compounds of the invention can be used to perform or provide any of the biological functions, such as modulating or inhibiting a biological substance or pathway having the same, described herein.

The subjects that can be treated with the compounds of the invention can be any animal, where humans are an example.

The inhibition or treatment provided by the compounds of the invention can be compared to the absence of the presence or administration of the compounds of the invention.

In previous studies, it was demonstrated that marmelin can inhibit the action of transcription factor NF-κB (Cancer Res. 2008 Oct. 15; 68(20): 8573-8581). The marmelin analogs were prepared as described herein, and tested to determine whether the analog compounds can bind to NF-κB. Based on computational docking studies, the analog compounds were determined to bind to the NF-κB p50 subunit (data not shown). The computer modeling docking studies showed that the binding of marmelin analogs into the active site of p50 subunit of NF-κB, where the naphthalene moiety is shown to interact with the active site of the NF-κB p50 subunit. As such, the core structure (e.g., naphthalene moiety) is retained in the analogs.

In addition, it was found that THB (i.e., MRL THB or MRLTHB) has better binding to the kinase domain of the cancer stem marker DCLK1 when compared to marmelin in computer modeling studies (data not shown). The computer modeling studies showed the binding of marmelin and THB into the active site of DCLK1 via the naphthalene moiety. This confirms that the THB analog binds with DCLK1 in the same manner as marmelin, and thereby the naphthalene moiety is retained in the analogs.

Studies were performed that demonstrate the anti-cancer effects of the analog compounds. Using hexoseaminidase assay, it was determined the compounds had an effect on cell proliferation. It was observed that THB is a potent inhibitor of dose and time dependent proliferation of both colon and pancreatic cancer cells (see Table 1).

Table 1 shows: THB being a potent inhibitor of proliferation of colon cancer cells; DHB (i.e., MRL DHB or MRLDHB) being a potent inhibitor of proliferation of pancreatic cancer cells; and THB being a potent inhibitor of proliferation in both colon pancreatic cancer cells. The IC50 value at 48 hours is 10 μM in both HCT116 and PanC-1 cells. The analog DHB is shown to inhibit proliferation of pancreatic cancer cells more effectively, and the IC50 values at 48 hours are 50 and 10 μM in both MiaPaCa-2 and PanC-1 cell lines, respectively. (Table 1).

Additionally, water-soluble derivatives of the analog compounds were prepared using β-cyclodextrin (i.e., CD). FTIR and DSC spectra analyses confirmed the identity of the compounds (data not shown). The FTIR and DCS spectra of CD, DHB, THB, and their beta-cyclodextrin inclusion complexes confirmed the complexes. In addition, scanning electron microscopic images with 10 μM and 5 μM concentrations confirmed that the CD complexes were water soluble. Also, H1-NMR spectra (data not shown) confirmed the water solubility. Further, relative host-guest geometry corresponding to the minimum of the energy of the formations of DHB and THB with beta-cyclodextrin (1:1) was identified by molecular docking, which showed that the CD complexes of DHB and THB still dock with the protein, and thereby can be functional similarly to the analog without the CD complex. Accordingly, the analog compounds can be mixed with CD to obtain a complex that is water soluble and can be included in a pharmaceutical composition for administration of the analog compounds.

Water solubility studies were performed with the CD/analog complexes. It was found that DHB, THB, and their cyclodextrin combinations—DHBCD and THBCD—inhibit proliferation of colon cancers, pancreatic cancers, and bladder cancers in a dose and time dependent manner, which is shown in Table 2. The observed IC50 value of THBCD at 48 h is below 10 μM in all the colon, pancreatic and bladder cancer cells.

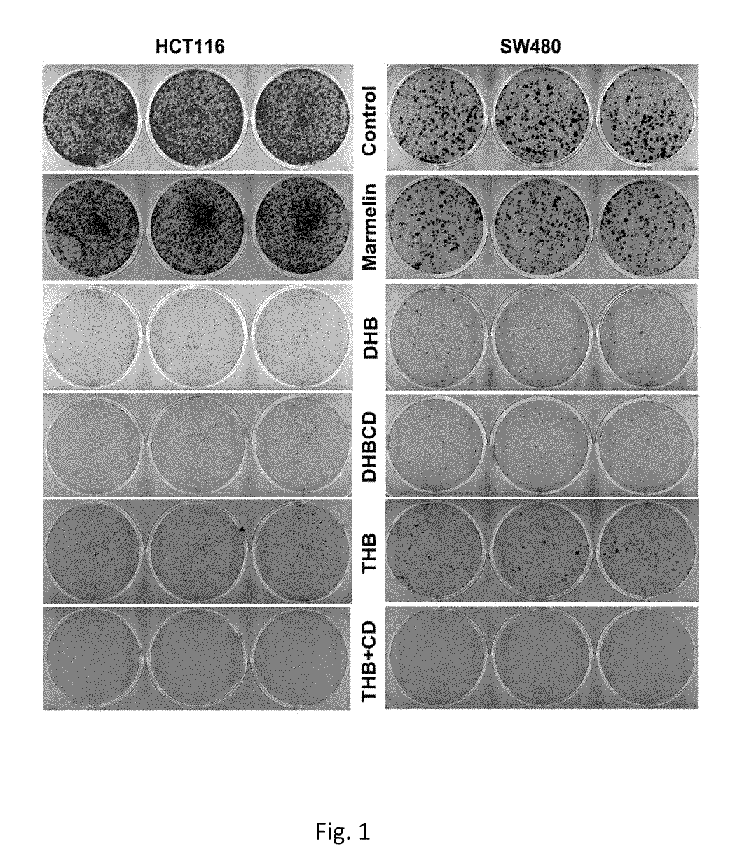

The analogs were tested to determine the ability to inhibit colony formation in both HCT116 and SW480 cells. FIG. 1 shows that marmelin does not appear to significantly inhibit colony formation in either of HCT116 and SW480 cells. On the other hand, DHB and THB significantly inhibited colony formation in both HCT116 and SW480 cells, and DHBCD (i.e., DHB+CD) and THBCD (i.e., THB+CD) showed even more significant inhibition of colony formation in both HCT116 and SW480 cells. Of the analogs and analog complexes (e.g., analog and CD) tested, it was determined that THBCD may be the most potent inhibitor of colony formation (FIG. 1).

The analogs were tested to determine the ability induce death of colon cancer cells. It was found that THBCD induces G2/M arrest, apoptosis and SubG0 cell death in both HCT116 and SW480 cells (Table 3).

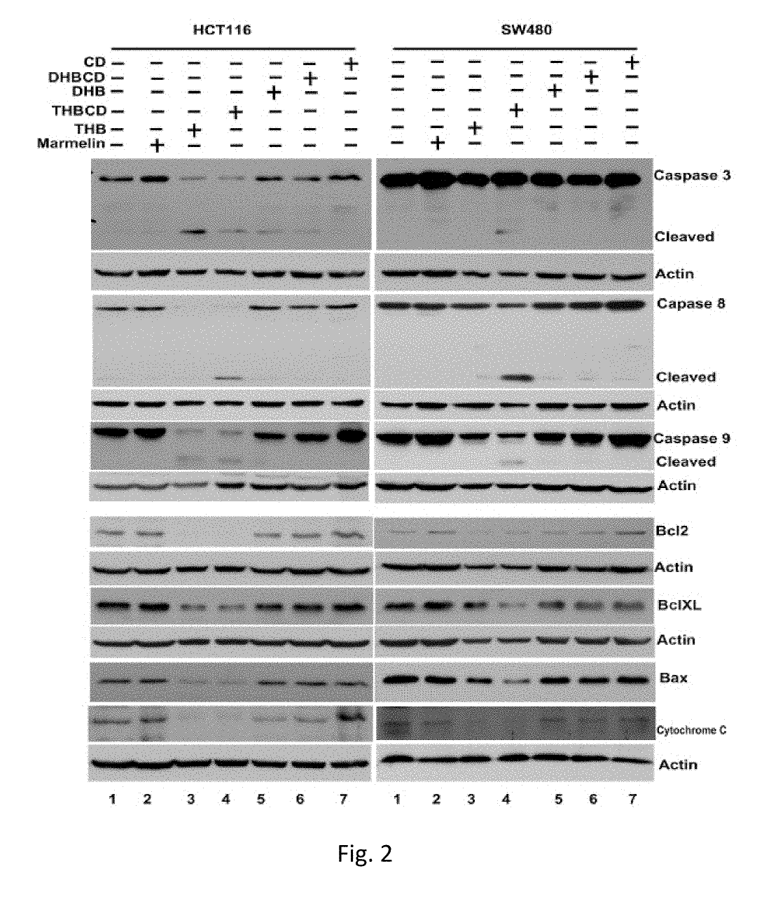

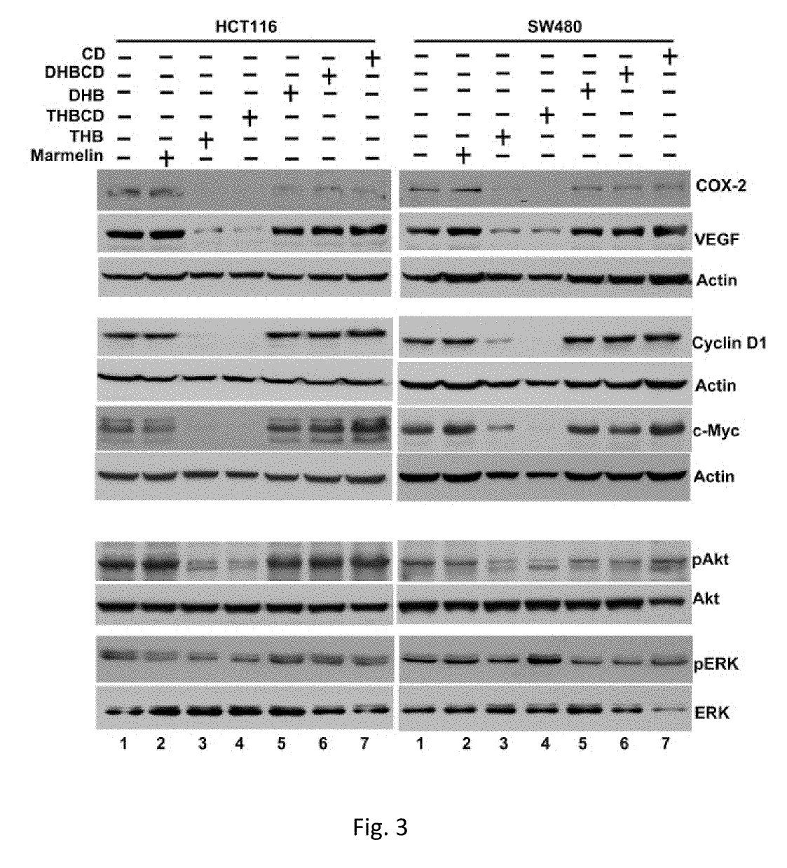

The pathways for apoptosis were studied to determine whether the analogs were capable of inhibiting certain aspects of the pathways. It was found that THBCD induces cell death in sub-G0 and apoptosis through the activation of caspase 3 in both cells, as shown in FIG. 2. FIG. 2 shows that THBCD is potent inducer of apoptosis in colon cancer cells. HCT116 and SW480 cells treated with MRL, THB, THBCD, DHB, DHBCD and CD for 48 h and performed for western blot analysis. THBCD induces apoptosis through activation of cleaved caspase 3 in both cells. It is shown that THBCD also activates caspase 8 and caspase 9. Furthermore, THB and THBCD inhibit anti-apoptotic protein Bcl2 and BclXL levels. THB and THBCD also inhibit cytochrome c suggesting that THBCD is a potent inducer of apoptosis. Moreover, THB and THBCD inhibit cyclin D1 and c-Myc levels suggest that it induces cell cycle arrest, as shown in FIG. 3. FIG. 3 shows that THBCD inhibits cancer promoting genes, Cyclin D1, c-Myc and Akt phosphorylation in colon cancer cells. HCT116 and SW480 cells treated with MRL, THB, THBCD, DHB, DHBCD and CD for 48 h and performed for western blot analysis. Furthermore, THB and THBCD inhibit phosphorylation of Akt (FIG. 3). In addition, these compounds inhibit cancer-promoting genes such as cyclooxygenase-2 (COX-2), and vascular endothelial growth factor (VEGF) expressions (FIG. 3).

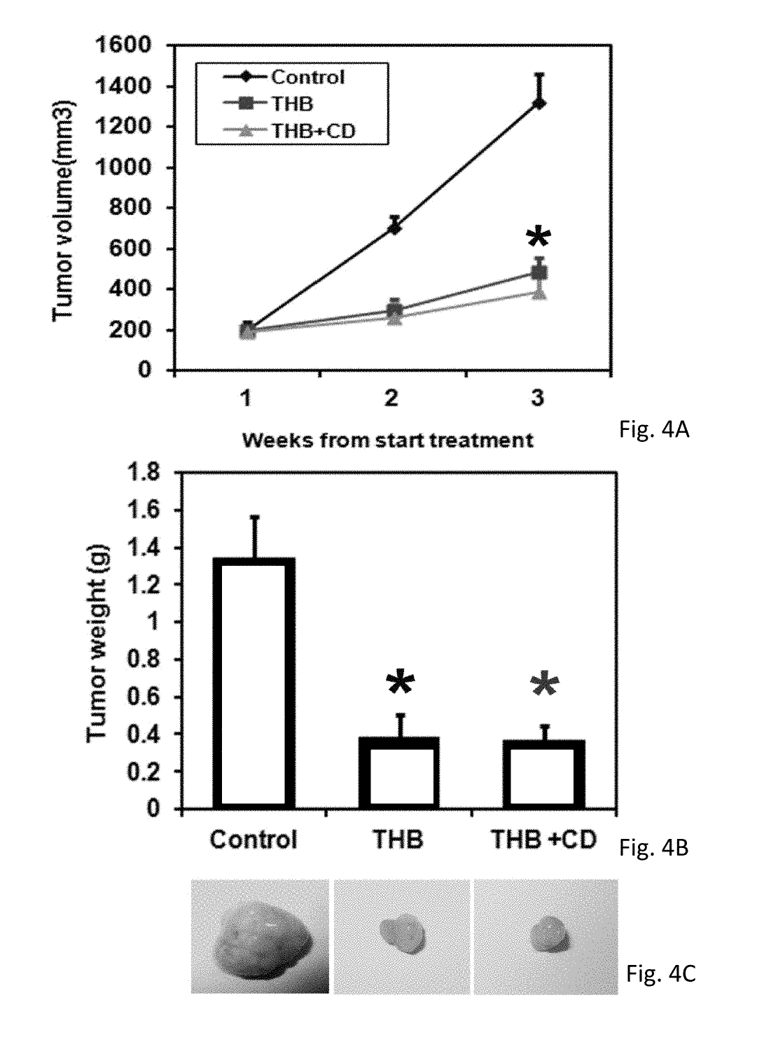

It was determined that THB and THBCD had an inhibiting effect on colon cancer tumor xenografts as shown in FIGS. 4A and 4B and 4C. For in vivo, HCT116 cells were injected into the blanks of nude mice, after one week when there was a palpable tumor and these compounds were injected intraperitoneally (5 mg/kg body weight) every day for 21 days. Tumor volumes were measured weekly. On 29^{th} day mice were euthanized and the tumors were removed and weighed for use in histology, immunohistochemistry, and gene expression studies. Both THB and THBCD inhibited growth of the tumor. In fact, tumor volume and weight were significantly reduced following treatment with THB and THBCD, as shown in FIGS. 4A-4C. It is noted that the images of FIG. 4C are from left to right: control; THB; and THBCD.

Moreover, it was found that THB and THBCD inhibit angiogenesis by CD31 staining (data not shown). The tumor tissues were used to perform immunohistochemistry and western blot analyses. THB and THBCD significantly reduced the COX-2, VEGF and cyclin D1 expression in tumor xenograft tissues in both immunohistochemistry and western blot analyses (data not shown). In addition, these compounds significantly reduced the phosphorylation of Akt in the tumor tissues (data not shown). Accordingly, it was found that THB and THBCD inhibit angiogenesis, where the tumor tissues were fixed with Zinc fixative and performed for immunohistochemistry analysis for CD31.

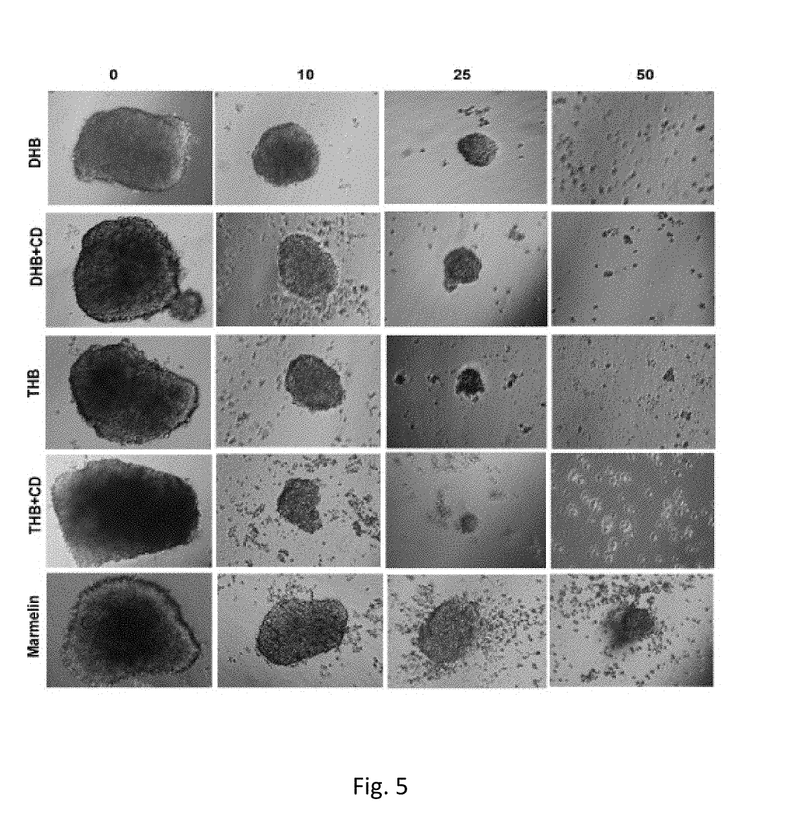

It was also determined the analogs inhibit colonosphere formation from cancer stem cells. Hence, the effect of the compounds on colonosphere formation was studied. The analogs that were studied (e.g., THB, THBCD, DHB, and DHBCD) significantly inhibited colonosphere formation, thereby suggesting that these analogs can inhibit cancer stem cells as shown in FIG. 5, such as from forming colonospheres. The columns are 0, 10, 25, and 50 micromolar concentrations of the compounds labeled by rows. As shown, both THB and THBCD are potent inhibitors of colonosphere formation.

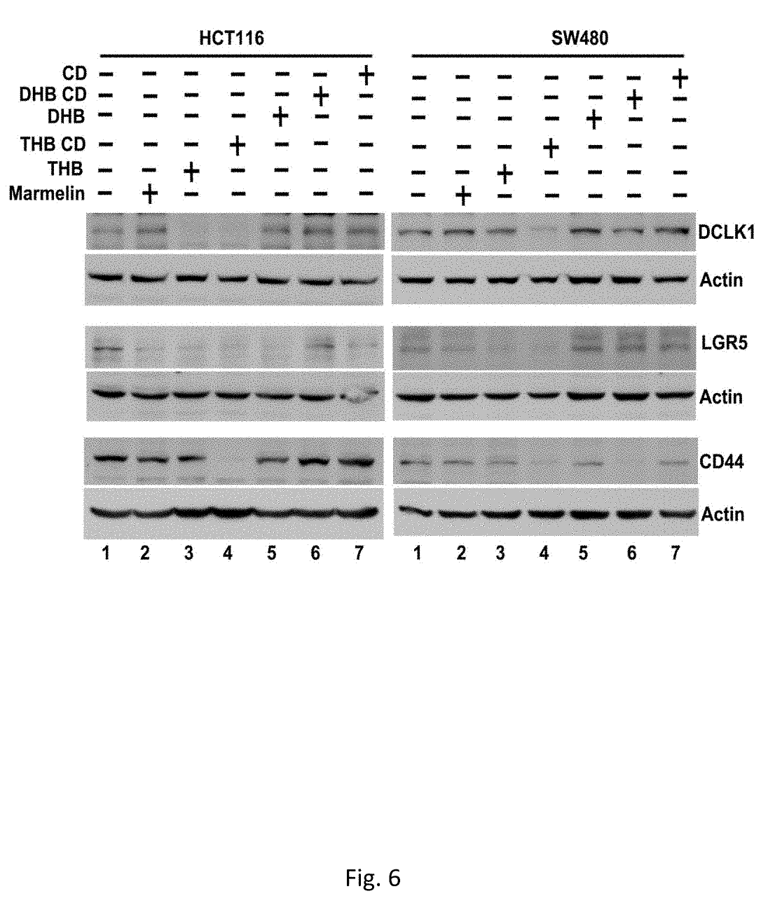

The analogs were also tested with doublecortin and CaM kinase-like-1 (DCLK1), a microtubule-associated kinase expressed in postmitotic neurons and is an intestinal stem cell marker that is expressed in colon adenocarcinoma. DCLK1 distinguishes between tumor and normal stem cells in the intestine and could be a therapeutic target for colon cancer. THB or THBCD treatment significantly inhibited the stem cell marker proteins DCLK1, LGR5 and CD44 expression in both HCT116 and SW480 cells as shown in FIG. 6.

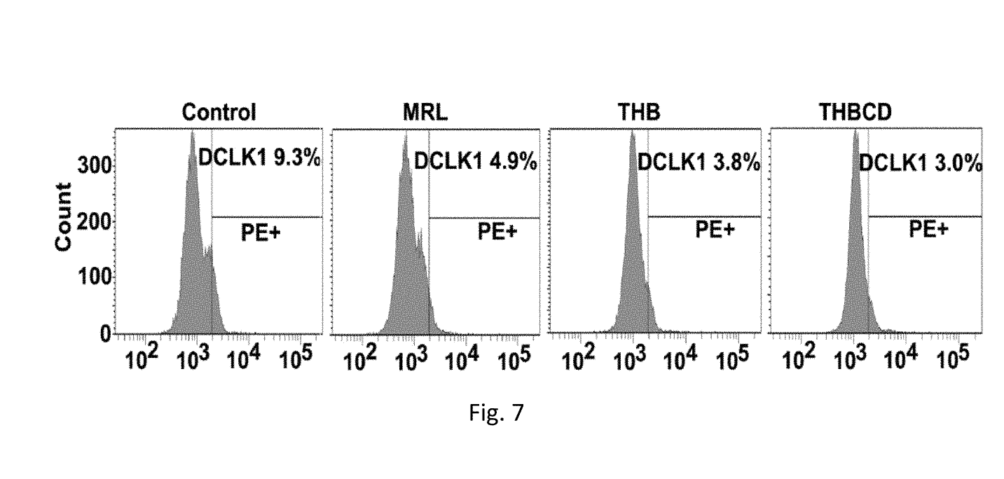

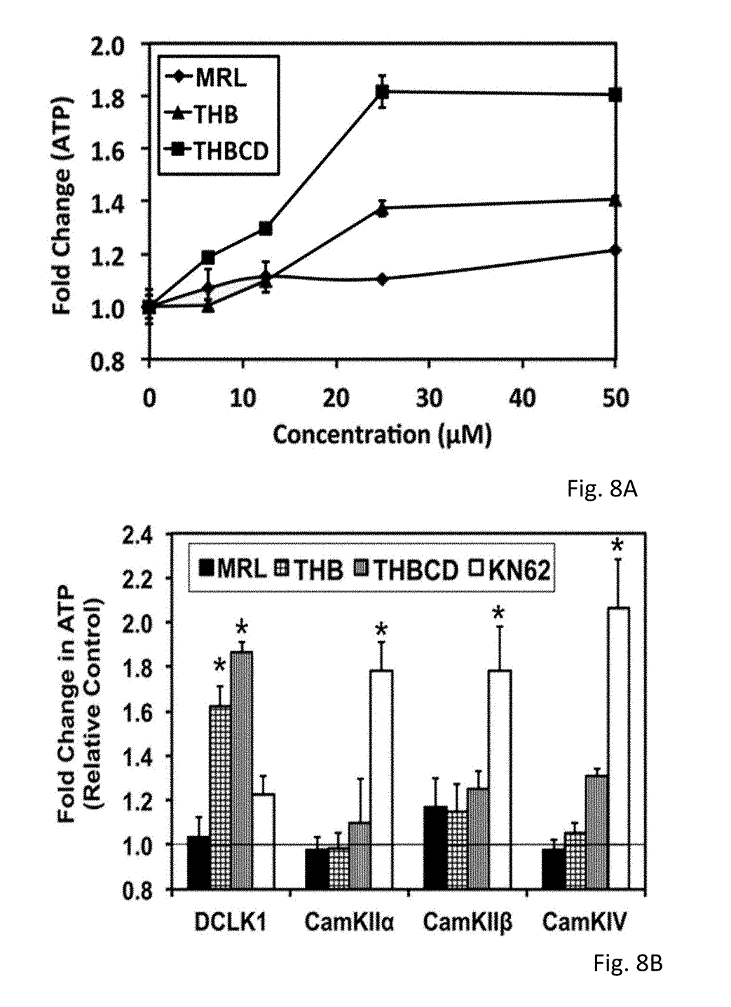

Additionally, flow cytometric analyses showed a significant decrease in DCLK1+ in HCT116 cells with THB or THBCD as shown in FIG. 7. DCLK1 encodes a calmodulin-like kinase domain, and has homology to calmodulin kinases CAMKII and CAMKIV. We performed homology modeling and determined that THB can interact with the kinase domain with binding energy of −5.94. Furthermore we performed an in vitro kinase assay using recombinant DCLK1. Inclusion of THB or THBCD significantly reduced the DCLK1 kinase activity in a dose dependent manner as shown in FIG. 8A. FIG. 8A shows THB and THBCD inhibit DCLK1 kinase activity. Also, THB and THBCD have 100-fold less activity against CAMKII and CAMKIV as shown in FIG. 8B suggesting that THB or THBCD is a specific competitive inhibitor of DCLK1 kinase activity. FIG. 8B shows THB and THBCD are specific competitive inhibitors of DCLK1 kinase activity. Furthermore, THB and THBCD inhibit cancer stem cell marker DCLK1 expression in the xenograft tumors in both immunohistochemistry and western blot analysis (data not shown).

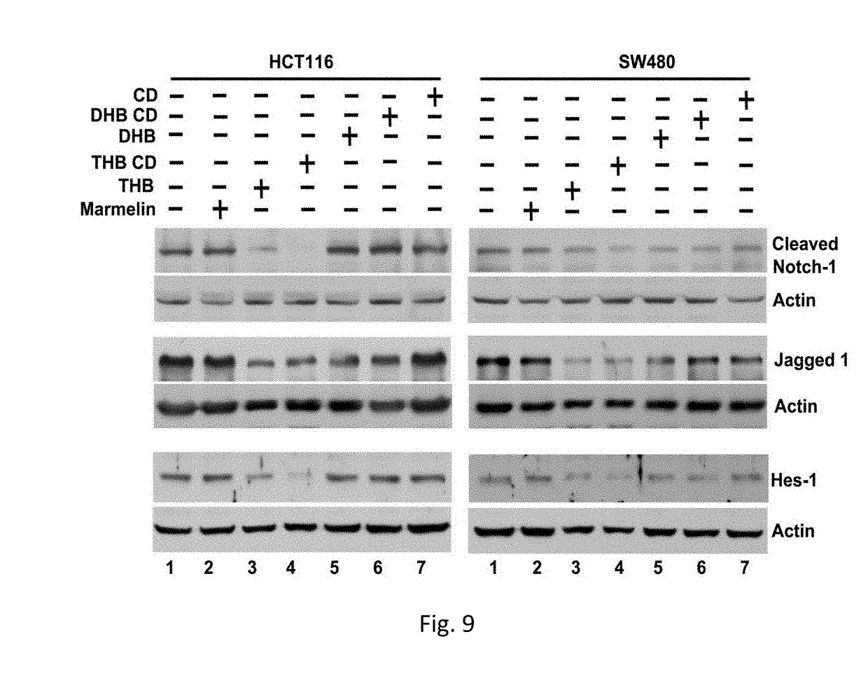

Notch signaling also plays a fundamental role in the differentiation and maintenance of stem cells. More importantly, altered Notch activity has been shown to partially explain the apparent radioresistance present in the stem cell fraction in cancers. This suggests that targeting the Notch signaling pathway might affect growth of cancer stem cells. We next determined the effect of THB or THBCD on Notch signaling-related proteins in the two colon cancer cells. Both Notch-1 and its ligand, Jagged-1 were downregulated by the THB or THBCD as shown in FIG. 9. Further confirmation was obtained when reduced expression of Hes-1 expression was observed (FIG. 9).

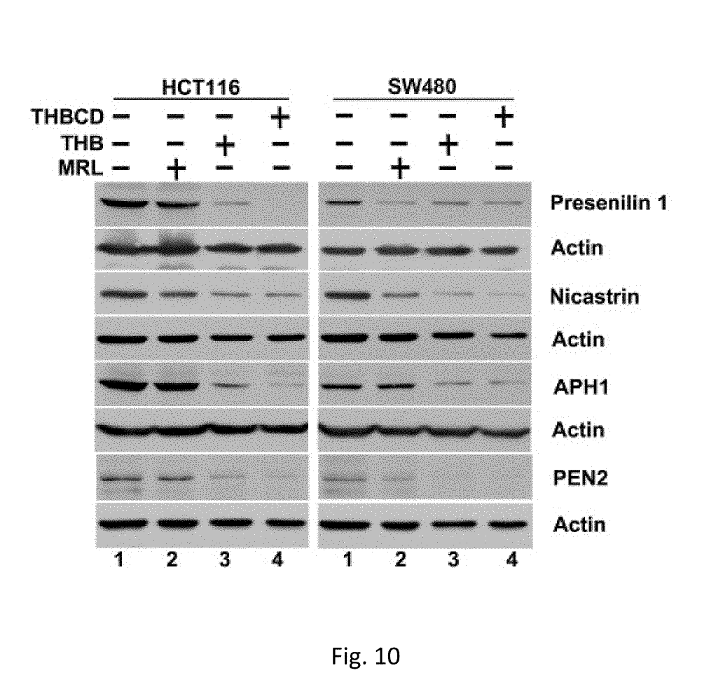

The γ-secretase enzyme complex is made up of four proteins presenilin, nicastrin, APH-1 (anterior pharynx-defective 1), and PEN-2 (presenilin enhancer 2), all of which are essential for activity. Cleavage by the γ-secretase complex releases the Notch intracellular domain (NICD), which in turn translocates into the nucleus of the cells, interacts with the C promoter-binding factor-1 (CBF1) transcriptional cofactor and transactivates target genes, such as those in the hairy and enhancer of split (Hes) and Hes related with YRPW motif (Hey) family proteins. We determined whether the γ-secretase complex comprising of Presenilin, Nicastrin, APH1 and PEN2 is affected. Treatment with THB or THBCD resulted in downregulation in the expression of all four proteins as shown in FIG. 10.

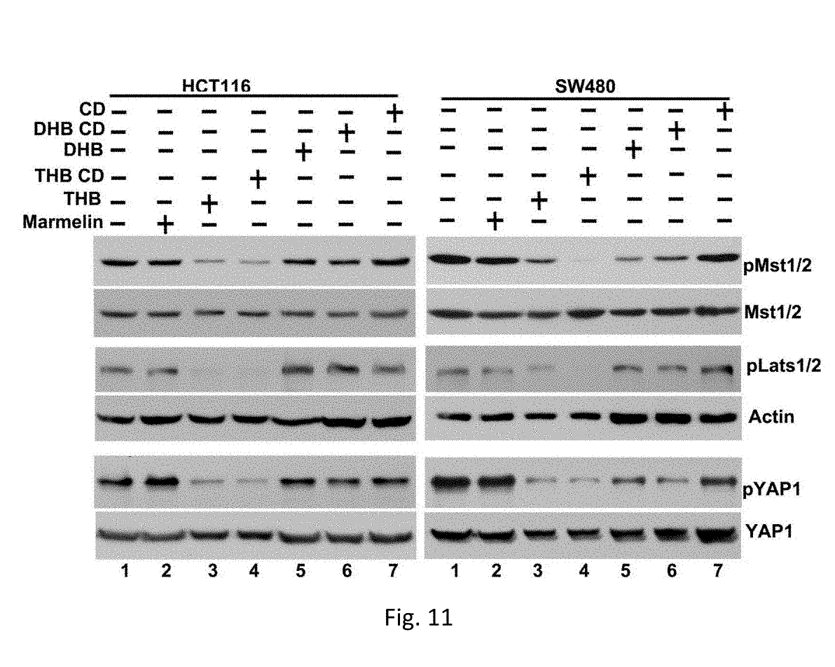

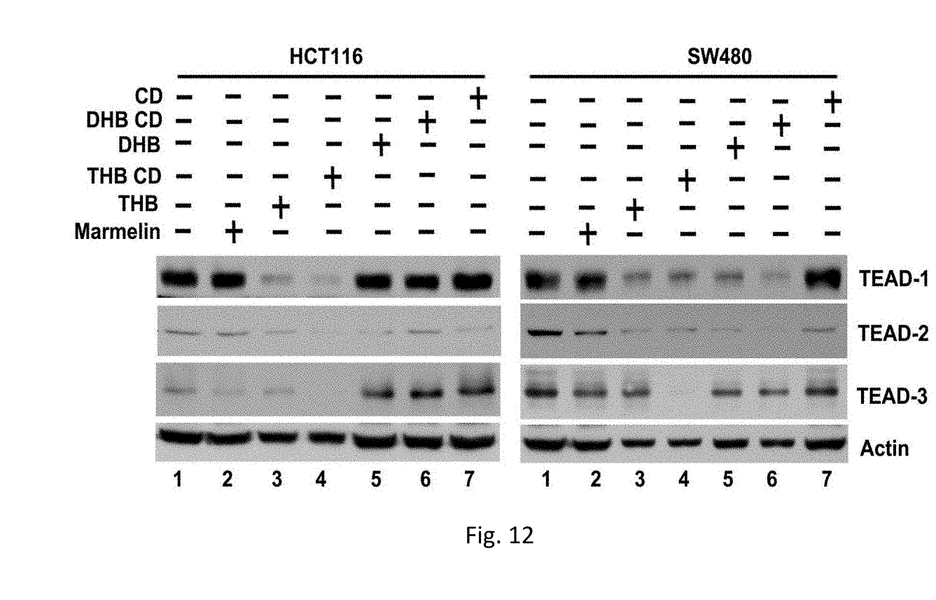

The Hippo signaling pathway YAP/TAZ/TEAD complex proteins have been found to be elevated in human cancers, including breast cancer, skin cancer, colorectal cancer, and liver cancer. The Hippo pathway regulates stem cell proliferation, self-renewal, and differentiation. YAP1 is highly expressed gene in stem cells. YAP1 stimulates Notch signaling, and administration of γ-secretase inhibitors suppressed the intestinal dysplasia caused by YAP1. We next determined the effect of THB or THBCD on Hippo signaling-related proteins in the two colon cancer cells. THB or THBCD treatment significantly downregulated the phosphorylation of Mst1/2, Lats1/2 and YAP1 in both HCT116 and SW480 cell lines as shown in FIG. 11. In addition, these compounds treatment resulted in significant downregulation in the expression of TEAD1, 2, and 4 as shown in FIG. 12. These data suggest that THB or THBCD downregulates the Hippo signaling pathway.

Overall, these data suggest that the novel derivatives of marmelin, THB and THBCD are potent anticancer agents that induce apoptosis, affect cancer stem cells and Notch & Hippo signaling pathways to inhibit tumor growth. Hence, the analogs have great potential as chemotherapeutic agents against colon cancer.

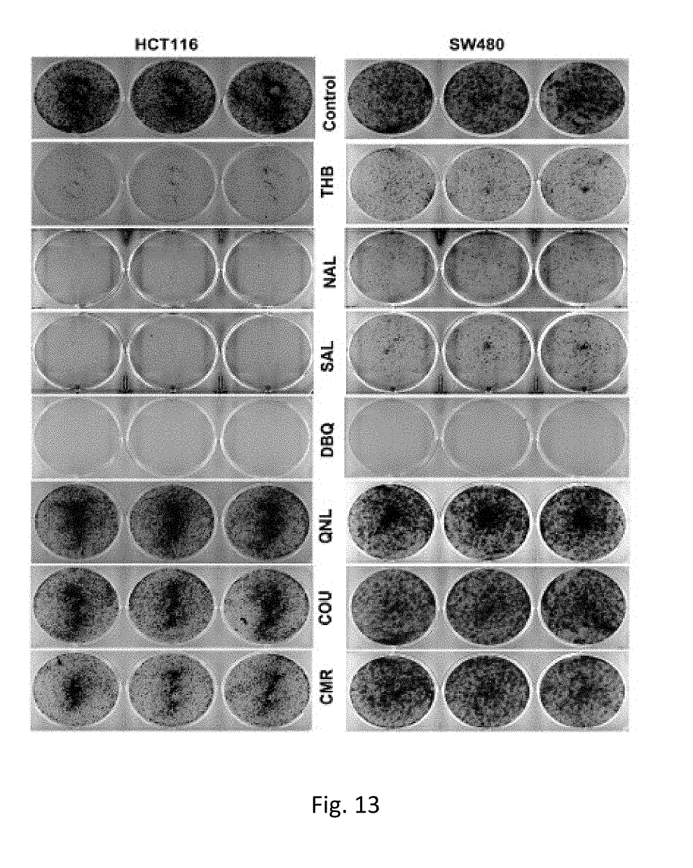

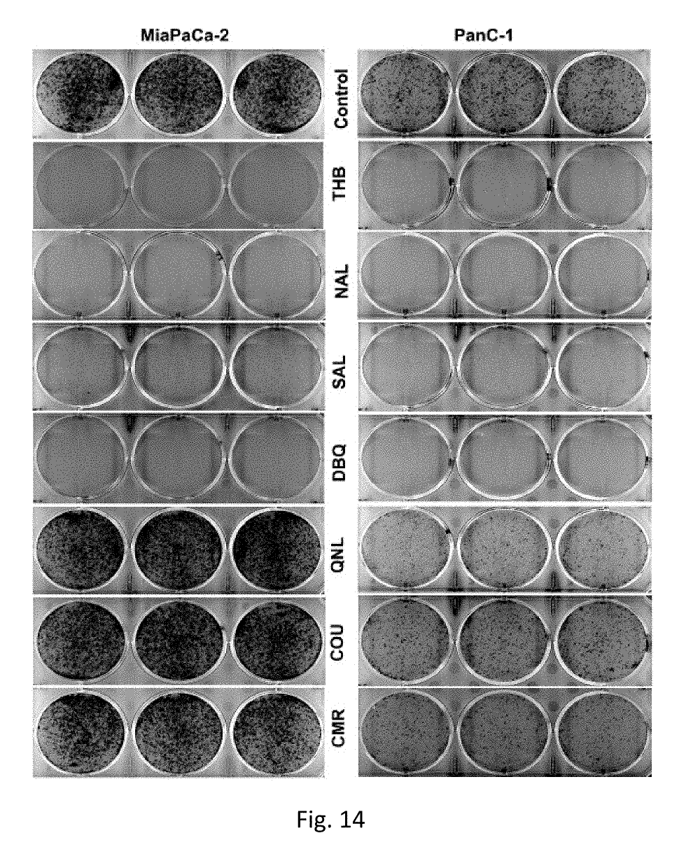

We have also performed studies to demonstrate the anti-cancer effects of the compounds: QNL (i.e., MRL QNL or MRLQNL), SAL (i.e., MRL SAL or MRLSAL), NAL (i.e., MRL NAL or MRLNAL), DBQ (i.e., MRL DBQ or MRLDBQ), COU (i.e., MRL COU or MRLCOU); and CMR (i.e., MRL CMR). Using hexoseaminidase assay, we determined the effect of the compounds on cell proliferation. We observed that NAL and DBQ are potent inhibitor of dose and time dependent proliferation of colon, pancreatic and bladder cancer cells Table 4. The IC50 value at 48 hours is below 10 μM in all the cancer cell lines. These three analogs, NAL, SAL and DBQ inhibit colony formation in all the four HCT116, SW480, MiaPaCa-2 and PanC-1 cells as shown in FIGS. 13-14. It is shown that DBQ is the most potent inhibitor of colony formation in all the four HCT116, SW480, MiaPaCa-2 and PanC-1 cells.

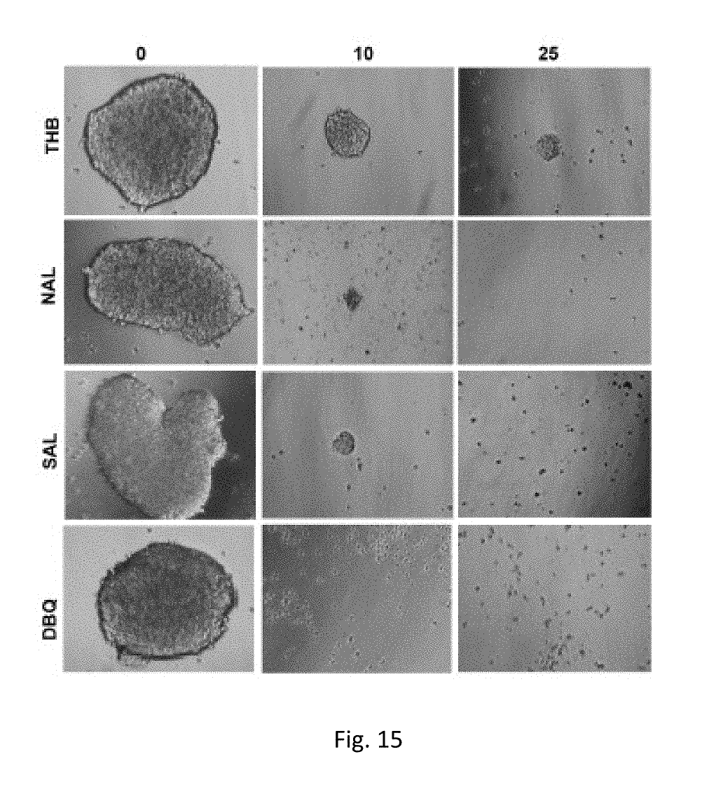

The effect of the compounds on colonosphere formation was determined. The analogs significantly inhibited colonosphere formation suggesting that it affect cancer stem cells FIG. 15. It can be seen, SAL, NAL and DBQ are potent inhibitors of colonosphere formation. Overall, the analogs have shown they can be used as chemotherapeutic agents against colon, pancreatic and bladder cancer.

HCT116 cells were treated with 5 μM and 10 μM of DBQ for 24 hours and then examined by flow cytometry following propidium iodide staining for DNA content (data not shown). Treatment with DBQ induces G2/M arrest in HCT116 cells.

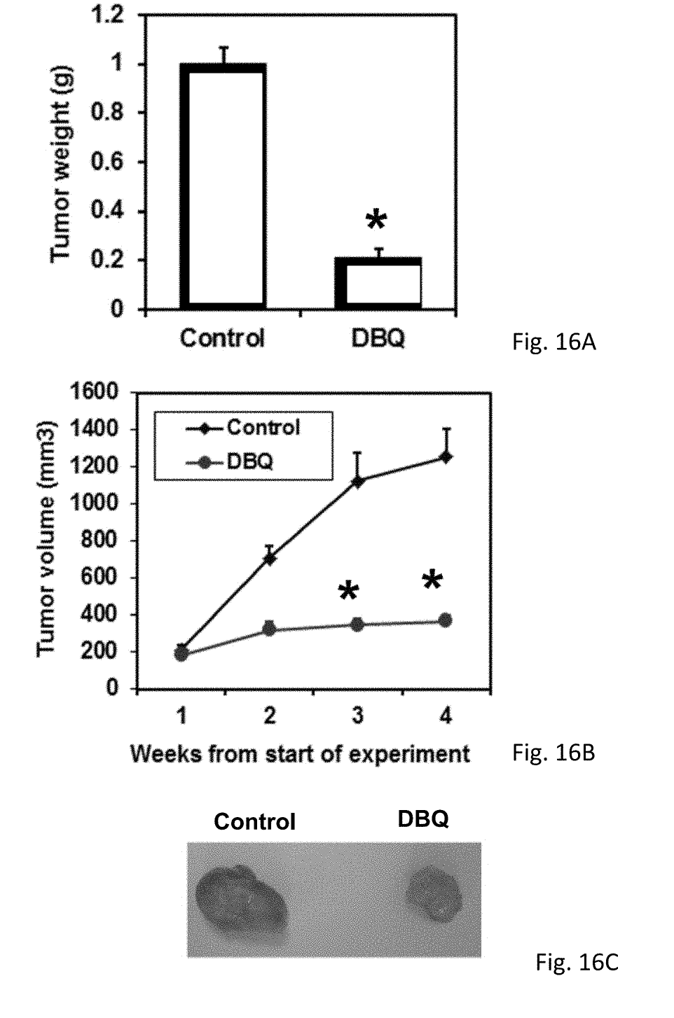

FIGS. 16A, 16B, and 16C show that DBQ can inhibit tumor growth. It was determined that DBQ had an inhibiting effect on colon cancer tumor xenografts. For in vivo, HCT116 cells were injected into the blanks of nude mice, after one week when there was a palpable tumor and these compounds were injected intraperitoneally (5 mg/kg body weight) every day for 21 days. Tumor volumes were measured weekly. On 29^{th} day mice were euthanized and the tumors were removed and weighed for use in histology, immunohistochemistry, and gene expression studies. DBQ inhibited the growth of the tumor. In fact, tumor volume and weight were significantly reduced following treatment with DBQ, as shown in FIGS. 16A-16C. It is noted that the images of FIG. 16C are from left to right: control; and DBQ.

We have also performed studies to demonstrate the anti-cancer effects of the compounds: MRL15, MRL16, MRL17, MRL18, MRL19, MRL20, MRL21, MRL22, MRL23, and MRL24. Using hexoseaminidase assay, we determined the effect of the compounds on cell proliferation. We observed that MRL16, MRL17, MRL18, MRL19, MRL20, MRL21, MRL23, and MRL24 are potent inhibitor of dose and time dependent proliferation of colon and pancreatic cancer cells Table 5. It was observed that MRL16, MRL17 and MRL 20 are potent inhibitor of dose and time dependent proliferation of colon and pancreatic cancer cells. The MRL16 IC50 value at 48 hours is below 0.3 μM in Colon cancer cell lines. These two analogs MRL 16 and 17 inhibit colony formation in HCT116 cells. Overall, the analogs can be used as chemotherapeutic agents against colon and pancreatic cancer.

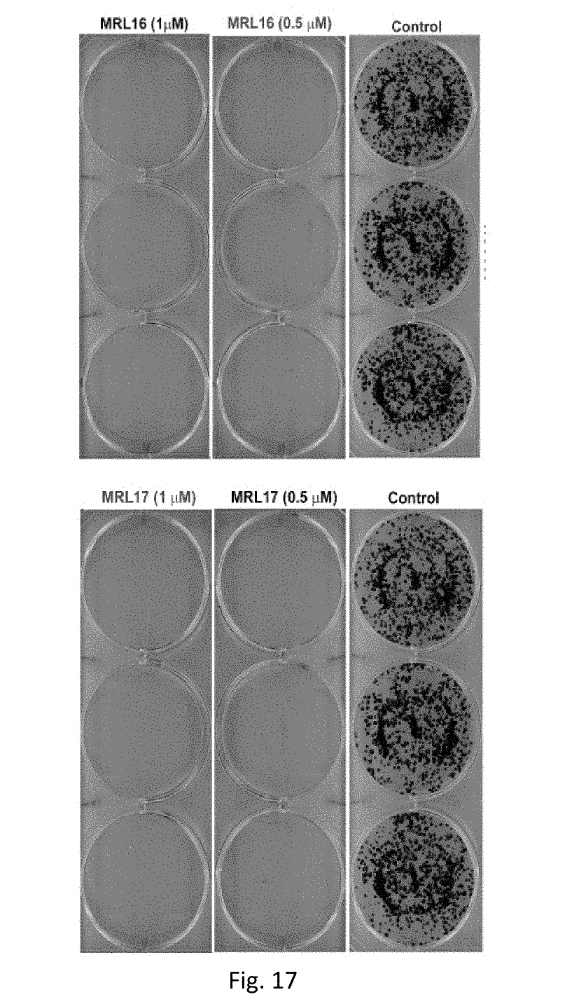

FIG. 17 shows that MRL16 and MRL 17 inhibit colony formation in colon cancer cell line HCT116.

Table 6 shows that MRL16&17 induces G2/M arrest in HCT116 cells. Table 6 also shows that MRL16 &17 induces G1 arrest in SW480 at 24 h and S-phase arrest at 48 h in SW480 cells.

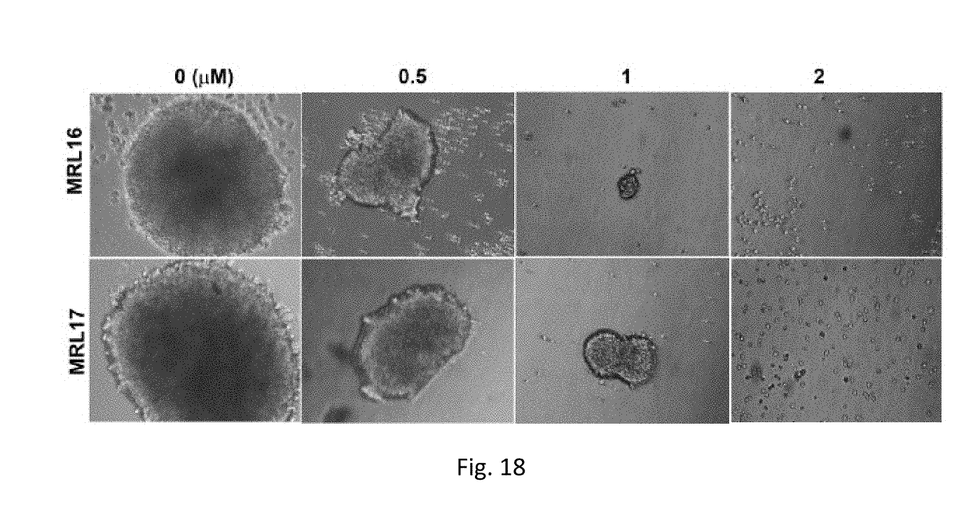

FIG. 18 shows that MRL16 and MRL17 inhibits colonosphere formation in HCT116 cells. These compounds may be preferred in some instances.

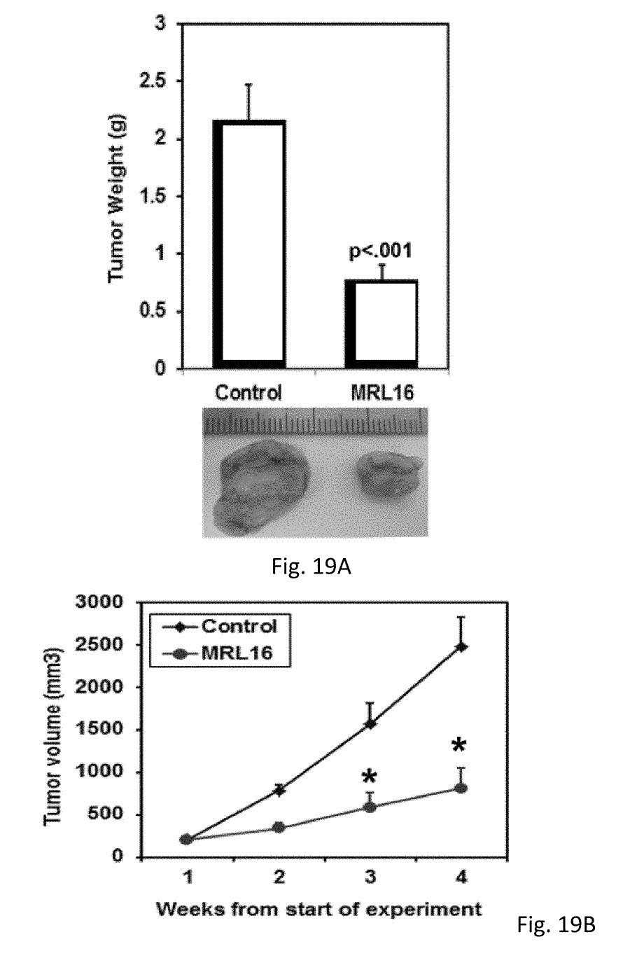

FIGS. 19A and 19B show MRL 16 inhibits tumor growth, such as on colon cancer tumor xenografts. For in vivo, HCT116 cells were injected into the blanks of nude mice, after one week when there was a palpable tumor and these compounds were injected intraperitoneally (2 mg/kg body weight) every day for 21 days. Tumor volumes were measured weekly. On 29th day mice were euthanized and the tumors were removed and weighed. MRL16 inhibited growth of the tumor. In fact, tumor volume and weight were significantly reduced following treatment with MRL16.

Marmelin analogs but not marmelin, can inhibit DCLK1 kinase activity. DCLK1 is an orphan kinase and this is the first specific inhibitor for the protein. Marmelin analogs are novel compounds that are 5 times more potent than its parent compound in inhibiting tumor growth. The cyclodextrin derivatives are also water soluble, which makes the compound easier for formulations. More importantly, the compounds not only affect dividing cancer cells, but also cancer stem cells. The biggest problem with the current approved chemotherapeutic agents is that these compounds only target fast dividing cancer cells and have not effect on stem cells. The compounds not only target the fast dividing cells, but are equally effective against cancer stem cells.

In addition to the compounds effects on cancer, there is an indication that the marmelin analogs can have efficacy against colonic inflammation.

The marmelin analogs may also be effective against other cancers such as breast, lung and osteosarcoma. This can allow for the compounds to be useful across a broad array of different cancers.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety:
1. Naishadham D, Lansdorp-Vogelaar I, Siegel R, Cokkinides V, Jemal A. State disparities in colorectal cancer mortality patterns in the United States. Cancer Epidemiol Biomarkers Prev. 2011; 20(7):1296-302.
2. Siegel R, Ward E, Brawley O, Jemal A. Cancer statistics, 2011: The impact of eliminating socioeconomic and racial disparities on premature cancer deaths. CA Cancer J Clin. 2011; 61(4):212-36.
3. Siegel R, Naishadham D, Jemal A. Cancer statistics, 2013. CA: a cancer journal for clinicians. 2013; 63(1):11-30.
4. Subramaniam D, Giridharan P, Murmu N, Shankaranarayanan N P, May R, Houchen C W, et al. Activation of apoptosis by 1-hydroxy-5,7-dimethoxy-2-naphthalene-carboxaldehyde, a novel compound from Aegle marmelos. Cancer research. [Research Support, N.I.H., Extramural Research Support, Non-U.S. Gov't]. 2008; 68(20):8573-81.

TABLE 1
IC 50 Values of MRL Analogs (μM Concentrations)

| | | Colon cancer cells | | | | | | Pancreatic cancer cells | | | | | |
|---|---|---|---|---|---|---|---|---|---|---|---|---|---|
| | MRL | HCT116 | | | SW480 | | | MiaPaCa-2 | | | PanC-1 | | |
| S. No | Analogs | 24 h | 48 h | 72 h | 24 h | 48 h | 72 h | 24 h | 48 h | 72 h | 24 h | 48 h | 72 h |
| 1. | MRL MHB | >50 | >50 | >50 | >50 | >50 | >50 | >50 | >50 | >50 | >50 | >50 | >50 |
| 2. | MRL DHB | >50 | >50 | 50 | >50 | >50 | 50 | >50 | 50 | 10 | >50 | 15 | 10 |
| 3. | MRL THB | >50 | 10 | 7.5 | >50 | 50 | 10 | >50 | 50 | 10 | >50 | 10 | 8 |
| 4. | MRL MFB | >50 | >50 | >50 | >50 | >50 | >50 | >50 | >50 | >50 | >50 | >50 | >50 |
| 5. | MRL RFB | 50 | 35 | 35 | 50 | 40 | 35 | >50 | 40 | 35 | >50 | 40 | 40 |
| 6. | MRL DFB | >50 | 25 | 22 | >50 | 20 | 18 | >50 | 25 | 17 | >50 | 50 | 20 |
| 7. | MRL TFB | >50 | >50 | >50 | >50 | >50 | >50 | >50 | >50 | >50 | >50 | >50 | >50 |
| 8. | MRL BDM | >50 | >50 | >50 | >50 | >50 | >50 | >50 | >50 | >50 | >50 | >50 | >50 |

TABLE 2
IC 50 Values of MRL Analogs DHB or THB conjugated with cyclodextrin (CD) (μM Concentrations)

| | | Colon cancer cells | | | | | | Pancreatic cancer cells | | | | | | Bladder cancer cells | | | | | |
|---|---|---|---|---|---|---|---|---|---|---|---|---|---|---|---|---|---|---|---|
| | MRL | HCT116 | | | SW480 | | | MiaPaCa-2 | | | PanC-1 | | | KU7 | | | 253JBV | | |
| S. No | Analogs | 24 h | 48 h | 72 h | 24 h | 48 h | 72 h | 24 h | 48 h | 72 h | 24 h | 48 h | 72 h | 24 h | 48 h | 72 h | 24 h | 48 h | 72 h |
| 1. | MRL | >50 | >50 | >50 | >50 | >50 | >50 | >50 | >50 | >50 | >50 | >50 | >50 | NA | NA | NA | NA | NA | NA |
| 2. | CD | >50 | >50 | >50 | >50 | >50 | >50 | >50 | >50 | >50 | >50 | >50 | >50 | NA | NA | NA | NA | NA | NA |
| 3. | DHB | >50 | >50 | 50 | >50 | >50 | 15 | >50 | 25 | 25 | >50 | 10 | 8 | NA | NA | NA | NA | NA | NA |
| 4. | DHBCD | >50 | >50 | 50 | >50 | >50 | 10 | >50 | 50 | 25 | >50 | 10 | 8 | NA | NA | NA | NA | NA | NA |
| 5. | THB | >50 | 10 | 8 | >50 | 10 | 9 | >50 | 10 | 9 | >50 | 10 | 8 | 22 | 8 | 6 | 50 | 38 | 15 |
| 6. | THBCD | >50 | 8 | 6 | >50 | 9 | 8 | >50 | 15 | 8 | >50 | 10 | 8 | >50 | 8 | 6 | >50 | 10 | 8 |

TABLE 3
Cell cycle analysis

| | | Colon cancer cells | | | | | | | |
|---|---|---|---|---|---|---|---|---|---|
| | | HCT116 | | | | SW480 | | | |
| | | SubG0 | | | | SubG0 | | | |
| | | Dead | | | | Dead | | | |
| | MRL | Cells | G0/G1 | | G2M | Cells | G0/G1 | S | G2M |
| S. No | Analogs | (%) | (%) | S (%) | (%) | (%) | (%) | (%) | (%) |
| 1. | Control | 26.5 | 47.7 | 11.8 | 12.6 | 2.8 | 58.9 | 14.5 | 21.0 |
| 2. | MRL | 29.9 | 45.3 | 11.5 | 11.4 | 6.0 | 65.3 | 10.2 | 18.1 |
| 3. | MRL DHB | 34.2 | 40.7 | 13.0 | 10.4 | 6.1 | 60.7 | 14.1 | 18.4 |
| 4. | MRL | 37.2 | 38.4 | 12.8 | 10.2 | 7.5 | 61.8 | 12.3 | 17.7 |
| | DHBCD | | | | | | | | |
| 5. | MRL THB | 29.0 | 17.2 | 28.1 | 19.7 | 10.8 | 39.5 | 21.8 | 26.8 |
| 6 | MRL | 38.6 | 35.2 | 19.6 | 5.9 | 36.8 | 47.6 | 12.6 | 2.9 |
| | THBCD | | | | | | | | |
| 7. | CD | 31.0 | 43.9 | 12.3 | 10.9 | 3.6 | 61.1 | 14.9 | 19.8 |

TABLE 4
IC 50 Values of MRL Analogs Series II (μM Concentrations)

| | | Colon cancer cells | | | | | | Pancreatic cancer cells | | | | | | Bladder cancer cells | | | | | |
|---|---|---|---|---|---|---|---|---|---|---|---|---|---|---|---|---|---|---|---|
| | MRL | HCT116 | | | SW480 | | | MiaPaCa-2 | | | PanC-1 | | | KU7 | | | 253JBV | | |
| S. No | Analog | 24 h | 48 h | 72 h | 24 h | 48 h | 72 h | 24 h | 48 h | 72 h | 24 h | 48 h | 72 h | 24 h | 48 h | 72 h | 24 h | 48 h | 72 h |
| 1. | QNL | >50 | >50 | 20 | >50 | >50 | >50 | >50 | >50 | 25 | >50 | >50 | >50 | NA | NA | NA | NA | NA | NA |
| 2. | SAL | 25 | 8 | 6 | >50 | >50 | 8 | >50 | >50 | 8 | >50 | 10 | 8 | NA | NA | NA | NA | NA | NA |
| 3. | NAL | 10 | 6 | 6 | 25 | 8 | 8 | 40 | 8 | 6 | 45 | 10 | 7 | 10 | 10 | 6 | >50 | 10 | 7 |
| 4. | DBQ | 10 | 5 | 5 | 10 | 6 | 5 | 25 | 8 | 6 | 45 | 9 | 7 | 20 | 8 | 6 | 50 | 25 | 6 |
| 5. | COU | >50 | >50 | >50 | >50 | >50 | >50 | >50 | >50 | >50 | >50 | >50 | >50 | NA | NA | NA | NA | NA | NA |
| 6. | CMR | >50 | >50 | >50 | >50 | >50 | >50 | >50 | >50 | >50 | >50 | >50 | >50 | NA | NA | NA | NA | NA | NA |

TABLE 5
IC 50 Values of MRL Analogs Series III (μM Concentrations)

| | MRL | Colon cancer cells | | | | | | Pancreatic cancer cells | | | | | |
|---|---|---|---|---|---|---|---|---|---|---|---|---|---|
| | Analogs | HCT116 | | | SW480 | | | MiaPaCa-2 | | | PanC-1 | | |
| S. No | Series III | 24 h | 48 h | 72 h | 24 h | 48 h | 72 h | 24 h | 48 h | 72 h | 24 h | 48 h | 72 h |
| 1. | MRL15 | >10 | >10 | >10 | >10 | >10 | >10 | >10 | >10 | >10 | >10 | >10 | >10 |
| 2. | MRL16 | >2 | 0.3 | 0.2 | >2 | 0.3 | 0.2 | >10 | >10 | 5 | >10 | 4 | 2.5 |
| 3. | MRL17 | >2 | 1 | 0.8 | >2 | 1 | 0.6 | >10 | >10 | 5 | >10 | 3 | 1 |
| 4. | MRL18 | >10 | 3 | 1 | >10 | 5 | 3 | >10 | >10 | 10 | >10 | 4 | 2 |
| 5. | MRL19 | >10 | 5 | 2.3 | >10 | >10 | 5 | >10 | >10 | >10 | >10 | 10 | 4 |
| 6. | MRL20 | >2 | 0.5 | 0.4 | >2 | 1.2 | 1 | >10 | 9 | 8 | >10 | 5 | 1 |
| 7. | MRL21 | 10 | 3.8 | 3 | >10 | 5 | 3 | >10 | 5 | 5 | >10 | 5 | 3 |
| 8. | MRL22 | >10 | >10 | >10 | >10 | >10 | >10 | >10 | >10 | >10 | >10 | >10 | >10 |
| 9. | MRL23 | >10 | >10 | >10 | >10 | >10 | >10 | >10 | 5 | 4 | >10 | >10 | >10 |
| 10. | MRL24 | >10 | 3.5 | 2 | >10 | 10 | 4 | >10 | >10 | 5 | >10 | 5 | 4 |

TABLE 6
Cell cycle analysis

| | | Colon cancer cells | | | | | | | | | | | | | | | |
|---|---|---|---|---|---|---|---|---|---|---|---|---|---|---|---|---|---|
| | | HCT116 | | | | | | | | SW480 | | | | | | | |
| | | 24 h | | | | 48 h | | | | 24 h | | | | 48 h | | | |
| | | SubG0 | | | | SubG0 | | | | SubG0 | | | | SubG0 | | | |
| | | Dead | G0/ | | | Dead | G0/ | | | Dead | G0/ | | | Dead | G0/ | | |
| | | Cells | G1 | S | G2/M | Cells | G1 | S | G2/M | Cells | G1 | S | G2/M | Cells | G1 | S | G2/M |
| # | Analog | (%) | (%) | (%) | (%) | (%) | (%) | (%) | (%) | (%) | (%) | (%) | (%) | (%) | (%) | (%) | (%) |
| 1. | Control | 1.23 | 63.6 | 16.9 | 18.0 | 2.26 | 63.0 | 15.6 | 18.4 | 0.65 | 51.4 | 21.3 | 26.3 | 0.74 | 63.2 | 15.0 | 20.9 |
| 2. | MRL16 | 1.11 | 57.3 | 14.5 | 26.9 | 1.70 | 63.7 | 14.4 | 19.9 | 0.77 | 59.8 | 19.7 | 19.7 | 1.05 | 58.4 | 20.0 | 20.1 |
| 3. | MRL17 | 0.99 | 63.9 | 14.0 | 20.9 | 1.57 | 63.1 | 14.3 | 20.8 | 0.50 | 54.9 | 22.3 | 22.0 | 1.12 | 59.3 | 20.8 | 18.3 |

1. A compound comprising:
a structure of Formula 7 or salt thereof, or stereoisomer thereof, or having any chirality at any chiral center, or tautomer, polymorph, solvate, or combination thereof: 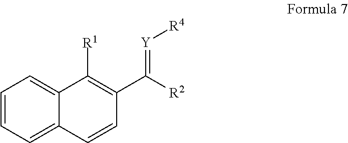
wherein:
R¹ or R² are each independently a substituent group;
Y is N; and
R⁴ includes a substituted polyaromatic.

2. The compound of claim 1, wherein:
R¹ or R² each independently includes one or more of a hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, thios, sulfhydryls, phosphors, carbonyls, carboxyls, amides, esters, amino acids, peptides, polypeptides, derivatives thereof, substituted or unsubstituted, or combinations thereof.

3. The compound of claim 1, wherein R¹ or R² each independently includes one or more of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₁-C₂₀ aryloxy, acyl, C₂-C₂₄ alkylcarbonyl (—CO-alkyl), C₆-C₂₀ arylcarbonyl (—CO-aryl), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X, wherein X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino (—CR═NH), alkylimino (—CR═N (alkyl), arylimino (—CR═N(aryl), nitro (—NO₂), nitroso (—NO), sulfo (—SO₂—OH), sulfonato (—S₂—O⁻), C₁-C₂₄ alkylsulfanyl (—S-alkyl), alkylthio, arylsulfanyl (—S-aryl), arylthio, C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O—)), phospho (—PO₂), phosphino (—PH₂), derivatives thereof, and combinations thereof whether substituted or unsubstituted or whether carbon backboned or having hetero atoms.

4. The compound of claim 2, wherein R¹ includes one or more of hydroxyl, halogen, or C₁-C₂₄ alkyl.

5. The compound of claim 4, wherein R² is a hydrogen or C₁-C₁₂ alkyl.

6. The compound of claim 5, wherein R² is a C₁-C₁₂ alkyl.

7. The compound of claim 5, wherein R² is methyl.

8. The compound of claim 7, wherein R¹ is a hydroxyl.

9. The compound of claim 1, wherein R⁴ includes a substituted naphthamide.

10. The compound of claim 9, wherein R⁴ includes a hydroxyl substituted naphthamide.

11. The compound of claim 10, wherein R⁴ includes a hydroxyl substituted 2-naphthamide.

12. The compound of claim 11, wherein R⁴ is:

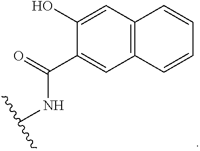 [SMILES: *NC(=O)c1cc2ccccc2cc1O]

13. The compound of claim 12, wherein R¹ includes one or more of hydroxyl, halogen, or C₁-C₂₄ alkyl.

14. The compound of claim 13, wherein R² is a hydrogen or C₁-C₁₂ alkyl.

15. The compound of claim 14, wherein R² is methyl.

16. The compound of claim 15, wherein R¹ is a hydroxyl.

17. The compound of claim 1 having the following structure or salt thereof, or stereoisomer thereof, or having any chirality at any chiral center, or tautomer, polymorph, solvate, or combination thereof:

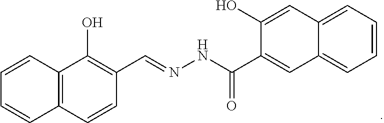 [SMILES: O=C(N/N=C/c1ccc2ccccc2c1O)c1cc2ccccc2cc1O]

18. The compound of claim 1 having the following structure or salt thereof, or stereoisomer thereof, or having any chirality at any chiral center, or tautomer, polymorph, solvate, or combination thereof:

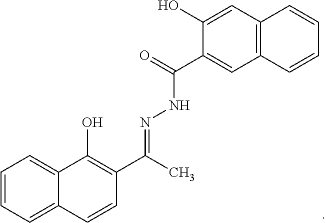 [SMILES: C/C(=N\NC(=O)c1cc2ccccc2cc1O)c1ccc2ccccc2c1O]

19. The compound of claim 1 having one of the following structures or salt thereof, or stereoisomer thereof, or having any chirality at any chiral center, or tautomer, polymorph, solvate, or combination thereof:

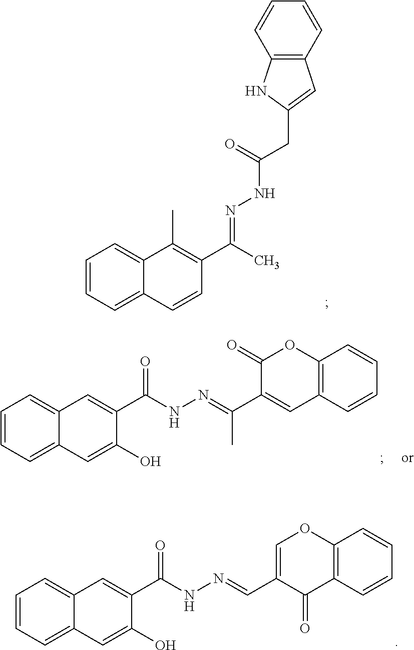 [SMILES: C/C(=N\NC(=O)Cc1cc2ccccc2[nH]1)c1ccc2ccccc2c1C ; C/C(=N\NC(=O)c1cc2ccccc2cc1O)c1cc2ccccc2oc1=O ; O=C(N/N=C/c1coc2ccccc2c1=O)c1cc2ccccc2cc1O]

20. A pharmaceutical composition comprising:
a compound of claim 1; and
a pharmaceutically acceptable carrier having the compound.
